SEQUENCE LISTING (6 sequences)

SEQ ID NO: 1 - DNA, 19 bp, Artificial
  Synthetic PCR primer.
actgaacccaaaggcacac

SEQ ID NO: 2 - DNA, 22 bp, Artificial
  Synthetic PCR primer.
aacatgacaatgaaggagctac

SEQ ID NO: 3 - DNA, 16 bp, Artificial
  Synthetic PCR primer.
gcctttacccattacc

SEQ ID NO: 4 - DNA, 18 bp, Artificial
  Synthetic PCR primer.
cccgcttatttttaatgc

SEQ ID NO: 5 - DNA, 2611 bp, Homo sapiens
gcgcgtccgggctcccggggctggtaatatagcggctcgccgaggcgctggtgcacgggggcagcgcgcagcaggccggcgggcaggcgggcgggctggctggcaggcaggactgggatcgaggcccagaaaacggagcagcgggcaccagggaggcctggaacggggcgagcgccatgagcaacaaatgcgacgtggtcgtggtggggggcggcatctcaggtatggcagcagccaaacttctgcatgactctggactgaatgtggttgttctggaagcccgggaccgtgtgggaggcaggacttacactcttaggaaccaaaaggttaaatatgtggaccttggaggatcctatgttggaccaacccagaatcgtatcttgagattagccaaggagctaggattggagacctacaaagtgaatgaggttgagcgtctgatccaccatgtaaagggcaaatcataccccttcagggggccattcccacctgtatggaatccaattacctacttagatcataacaacttttggaggacaatggatgacatggggcgagagattccgagtgatgccccatggaaggctccccttgcagaagagtgggacaacatgacaatgaaggagctactggacaagctctgctggactgaatctgcaaagcagcttgccactctctttgtgaacctgtgtgtcactgcagagacccatgaggtctctgctctctggttcctgtggtatgtgaagcagtgtggaggcacaacaagaatcatctcgacaacaaatggaggacaggagaggaaatttgtgggcggatctggtcaagtgagtgagcggataatggacctccttggagaccgagtgaagctggagaggcctgtgatctacattgaccagacaagagaaaatgtccttgtggagaccctaaaccatgagatgtatgaggctaaatatgtgattagtgctattcctcctactctgggcatgaagattcacttcaatccccctctgccaatgatgagaaaccagatgatcactcgtgtgcctttgggttcagtcatcaagtgtatagtttattataaagagcctttctggaggaaaaaggattactgtggaaccatgattattgatggagaagaagctccagttgcctacacgttggatgataccaaacctgaaggcaactatgctgccataatgggatttatcctggcccacaaagccagaaaactggcacgtcttaccaaagaggaaaggttgaagaaactttgtgaactctatgccaaggttctgggttccctagaagctctggagccagtgcattatgaagaaaagaactggtgtgaggagcagtactctgggggctgctacacaacttatttcccccctgggatcctgactcaatatggaagggttctacgccagccagtggacaggatttactttgcaggcaccgagactgccacacactggagcggctacatggagggggctgtagaggccggggagagagcagcccgagagatcctgcatgccatggggaagattccagaggatgaaatctggcagtcagaaccagagtctgtggatgtccctgcacagcccatcaccaccacctttttggagagacatttgccctccgtgccaggcctgctcaggctgattggattgaccaccatcttttcagcaacggctcttggcttcctggcccacaaaagggggctacttgtgagagtctaaagagagagggtgtctgtaatcacactctcttcttactgtatttgggatatgagtttggggaaagagttgcagtaaagttccatgaagacaaatagtgtggagtgaggcggggagcatgaagataaatccaactctgactgtaaaatacatggtatctctttctccgttgtggcccctgcttagtgtcccttacctggcttagcgttctgtttcaccagtttccaagtttattgccctcaaaatctttagaatagttaaattggcttgtttaaggttcttgctgccccacaacacaccttgcccatgcacaaggaatgaattttttcctaccattatggctttgtgcttgttcttcctcttacctgtaatagcctcaccttccctagttctttgcattcgtccttagaatactgtattgttacagctgaaagacagtaaagaccatttagtcctcaccttctgttttagagttgagcaaactgaagcccacagaggtggaacttaattacctaagagccacaataagccactggtatctgggggactagaacacaaatccaacgcttttcccacctctttggatgttttccccaattatcctccttcactccctgtcatagttaccgatggtgtcccgttgtgtgggtttactctgtgctaagttgtcttacacttctcaaatgctactcagtatatagccttaagtcttactgttttgtgcggtgtgtctccagctgattttaacttttttgatggtagaaattttatctcttcttccttttgtatcctccattgtatcttcatacaaaggacagtacacacttgggtaattaaaaataaaagttgattgaccataaaaaaaaaaaaaaaaa

SEQ ID NO: 6 - protein, 520 aa, Homo sapiens
MSNKCDVVVVGGGISGMAAAKLLHDSGLNVVVLEARDRVGGRTYTLRNQKVKYVDLGGSYVGPTQNRILRLAKELGLETYKVNEVERLIHHVKGKSYPFRGPFPPVWNPITYLDHNNFWRTMDDMGREIPSDAPWKAPLAEEWDNMTMKELLDKLCWTESAKQLATLFVNLCVTAETHEVSALWFLWYVKQCGGTTRIISTTNGGQERKFVGGSGQVSERIMDLLGDRVKLERPVIYIDQTRENVLVETLNHEMYEAKYVISAIPPTLGMKIHFNPPLPMMRNQMITRVPLGSVIKCIVYYKEPFWRKKDYCGTMIIDGEEAPVAYTLDDTKPEGNYAAIMGFILAHKARKLARLTKEERLKKLCELYAKVLGSLEALEPVHYEEKNWCEEQYSGGCYTTYFPPGILTQYGRVLRQPVDRIYFAGTETATHWSGYMEGAVEAGERAAREILHAMGKIPEDEIWQSEPESVDVPAQPITTTFLERHLPSVPGLLRLIGLTTIFSATALGFLAHKRGLLVRV

(19) United States
(12) Patent Application Publication
(10) Pub. No.: US 2011/0067123 A1
(43) Pub. Date: Mar. 17, 2011

(54) MAO-B ELEVATION AS AN EARLY PARKINSON'S DISEASE BIOMARKER

(76) Inventors: Julie Andersen, Novato, CA (US); Jyothi Kumar Mallajosyula, Carlsbad, CA (US)

(21) Appl. No.: 12/867,233

(22) PCT Filed: Feb. 18, 2009
(86) PCT No.: PCT/US09/34416

§ 371 Date: Nov. 22, 2010

Related U.S. Application Data
(60) Provisional application No. 61/029,749, filed on Feb. 19, 2008.

(51) Int. Cl. A01K 67/027; C12Q 1/68; C40B 30/04; G01N 33/573; C12Q 1/26; A61K 31/137; A61K 31/135; A61K 31/44; A61K 31/4245; A61K 31/536; A61K 31/16; A61P 25/16

(52) U.S. Cl. 800/9; 435/6; 506/9; 435/7.4; 435/25; 514/654; 514/655; 514/657; 514/354; 514/364; 514/230.5; 514/626

(57) ABSTRACT

This invention pertains to development of a new animal model for Parkinson's Disease (PD) and to the discovery that elevated monoamine oxygenase B (MOA-B) expression and/or activity is a strong prognostic indicator for the disease. Accordingly, in certain embodiments, methods are provided for identifying a mammal at risk for Parkinson's disease. The methods typically involve determining level of expression or activity of monoamine oxidase B (MAO-B) in a sample from the mammal wherein an elevated level of MAO-B expression and/or activity as compared to a control (reference) is an indicator that the mammal has an increased likelihood of developing Parkinson's disease.

### CROSS-REFERENCE TO RELATED APPLICATIONS

This application claims benefit of and priority to U.S. Ser. No. 61/029,749, filed on Feb. 19, 2008, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported in party by Gran No: R01 NS_{—}045615 from the National Institutes of Health. The government of the United States of America has certain rights in this invention.

### FIELD OF THE INVENTION

This invention pertains to the field of prognostic assays and preventive medicine. In particular, this invention pertains to the discovery that elevated MAO-B expression or activity is a prognostic indicator of increased likelihood for Parkinson's disease.

### BACKGROUND OF THE INVENTION

Monoamine oxidase B (MAO-B) is found in the brain primarily in non-neuronal cells such as astrocytes and radial glia (Westlund et al. (1988) Neuroscience 25: 439-456; Westlund et al. (1985) Science 230: 181-183; Levitt et al. (1982) Proc. Natl. Acad. Sci., USA, 79: 6385-6389). Its levels are known to increase with age and in association with neurodegenerative disease in both humans and mice (Saura et al. (1994) J Neural Transm Suppl 41: 89-94; Fowler et al. (1980) J Neural Transm 49: 1-20; Riederer et al. (1987) Adv Neurol 45: 111-118; Gerlach et al. (1996) Neurology 47: S137-145). Substrate oxidation by the enzyme is accompanied stoichiometrically by the reduction of oxygen to H₂O₂ (Vindis et al. (2001) Kidney Int 59: 76-86; Cohen et al. (1997) Proc. Natl. Acad. Sci., USA, 94: 4890-4894). It has been postulated that age-related increases in MAO-B activity may contribute to cellular degeneration in the brain due to corresponding increases in the production of this reactive oxygen species (ROS) (Adams and Odunze (1991) Free Radic Biol Med 10: 161-169). Although MAO-B is expressed primarily in astrocytes and not directly within dopaminergic cells, H₂O₂ has a high membrane permeability and therefore it can induce toxic effects not only within the cell of origin, but also in neighboring cells (Halliwell (1992) J Neurochem 59: 1609-1623). The area of the brain preferentially impacted in Parkinson's disease (PD), the substantia nigra (SN), contains high levels of MAO-B positive astrocytes which are themselves somewhat protected against the effects of H₂O₂ due to the fact that they contain high levels of both GS11 and glutathione peroxidase which act in concert to detoxify H₂O₂ within cells (Halliwell (1992) J Neurochem 59: 1609-1623; Raps et al. (1989) Brain Res 493: 398-401; Sagara et al. (1993) J Neurochem 61: 1672-1676; Makar et al. (1994) J Neurochem 62: 45-53; Kang et al. (1997) Neuroreport 8: 2053-2060). Neurons, which contain significantly lower levels of these protective components, are particularly vulnerable to this mild oxidizing agent (Buckman et al. (1993) J Neurochem 60: 2046-2058; Behl et al. (1994) Cell 77: 8 17-827; Whittemore et al. (1994) Neuroreport 5: 1485-1488). This suggests that H₂O₂ produced within astrocytes by MAO-B may be either broken down to H₂O₂ within these cells or may diffuse to vulnerable nearby cells such as dopaminergic neurons (Wei et al. (1996) J Neurosci Res 46: 666-673). MAO-B activity levels have been found to be doubled in the SN in Parkinson's disease, and to correlate with the percentage of dopaminergic SN cell loss (Damier et al. (1996) Neurology 46: 1262-1269).

MAO-B-catalyzed ROS production has been suggested to contribute to an age-related increase in mitochondrial damage particularly in the SN (Soong et al. (1992) Nat Genet 2: 318-323). Selective reductions in complex I activity are associated with PD and selective inhibition of complex I via systemic administration of either rotenone or 1-methyl-4-phenyl-1,2,3,6-tetrahydropyridine (MPTP) results in patterns of morphological damage in rodents similar to that observed in the human Parkinsonian midbrain (Betarbet et al. (2000) Nat Neurosci 3: 1301-1306; Przedborski and Vila (2003) Ann N Y Acad Sci 991: 189-198). MAO-B is responsible for conversion of MPTP to MPP⁺ within astrocytes from where MPP⁺ can diffuse extracellularly. From there, it can be selectively transported into dopaminergic neurons via the dopamine transporter and elicit inhibitory effects on mitochondrial complex I activity (Gainetdinov et al. (1997) J Neurochem 69: 1322-1325).

There are numerous reports demonstrating the neuroprotective effects of the MAO-B inhibitors deprenyl or selegiline in Parkinsonian animal models although in many cases neuroprotection has been attributed to either antioxidant or antiapoptotic properties of the parent compounds or their metabolites (Wu et al. (1996) Ann N Y Acad Sci 786: 379-390; Magyar and Szende (2004) Neurotoxicology 25: 23 3-242; Szende et al. (2001) J Neural Transm 108: 25-33). Several multi-center studies including DATATOP, addressing the efficacy of selegiline administration in Parkinson's disease (PD) patients have concluded that selegiline both delayed and reduced the requirement of L-DOPA supplementation in early stages of symptomatic PD (Shoulson (1992) The Parkinson Study Group. Eur Neurol 32 Suppl 1: 46-53; LeWitt (1994) J Neural Transm Suppl 43: 171-181; Schneider (1995) J Neural Transm Suppl 46: 39 1-397) but had no effect on the mortality rate (Parkinson_Study_Group (1998) Ann Neurol 43: 3 18-325). Although deprenyl was found to be ineffective in slowing or halting the ultimate mortality in clinical PD it was given to patients only after symptoms were present which is known to coincide with the presence of an already extensive (˜60%) midbrain dopaminergic cell loss (Fearnley and Lees (1991) Brain 114 (Pt 5): 2283-2301). These studies suggest that inhibition of MAO-B activity at this advanced stage in the disease is ineffective.

### SUMMARY OF THE INVENTION

The experiments described herein were to test whether or not MAO-B elevation is a factor in initial neuropathology associated with Parkinson's disease and whether MAO-B inhibitors if given pre-symptomatically might be protective. In order to test the hypothesis that elevations in MAO-B can directly contribute to pathologies observed in PD and to better understand the possible mechanisms underlying its effects on these parameters, genetically engineered mouse lines in which MAO-B levels can be inducibly increased specifically within astrocytes in adult animals were created. This facilitated studies eliminating the impact of increased MAO-B expression during the developmental time period of the test animals.

It was discovered that astrocytic elevation of MAO-B in adult mice recapitulates many of the pathological hallmarks associated with human PD and that these effects can be prevented by treatment with the selective MAO-B inhibitor deprenyl or the antioxidant EUK1 89.

Thus, in certain embodiments this invention pertains to the discovery that monoamine oxidase B (MAO-B) expression or activity is a prognostic indicator of increased likelihood or susceptibility for the development of Parkinson's disease. Accordingly, in certain embodiments, methods are provided of identifying a mammal (e.g., a human) at risk for Parkinson's disease. The methods typically involve determining a level of expression and/or activity of monoamine oxidase B (MAO-B) in the mammal or in a sample from the mammal where an elevated level of MAO-B expression and/or activity, e.g., as compared to a control is an indicator that the mammal has an increased likelihood of developing Parkinson's disease. In certain embodiments the mammal is asymptomatic for Parkinson's disease. In certain embodiments the mammal presents with no significant neuronal cell loss. In certain embodiments the mammal is a human (e.g., infant, child, adolescent, or adult) that presents without symptoms of Parkinson's disease, but is believed to be at risk for the disease. In certain embodiments the method involves the sample is a sample that comprises one or more biological materials such as blood or a blood fraction, platelets, saliva, cerebrospinal fluid, a tissue sample (e.g., a neural tissue sample), a cell sample, and the like. In various embodiments the control comprises an expression and/or activity level determined for a population of the mammal that does not develop Parkinson's disease. In certain embodiments the population consists of members of the same species having the same age, and/or sex, and/or ethnicity as the subject being assayed. In certain embodiments the control comprises a threshold value or range designated as indicative of increased risk for Parkinson's disease. In certain embodiments the control is the level of MAO-B expression and/or activity in same subject at a different time in their life. In certain embodiments the control value is the threshold of top 30%, 25%, 20%, 10%, 5%, or 1% percentile of the full range of MAO-B activity within a random population. In certain embodiments the method is a component of a differential diagnosis for Parkinson's disease. In certain embodiments the method further comprises recording the determined level of MAO-B expression or activity, and/or a diagnosis based at least in part on the determined level of MAO-B expression or activity, in a patient medical record (e.g., hospital medical record, a doctor's office medical record, an insurance company medical record, a health maintenance organization (hmo) medical record, a personal medical record, a laboratory medical record, a personal medical record website, a computer readable medium storing a medical record, and a radio frequency tag (RF tag). In certain embodiments a diagnosis, based at least in part on the level of expression or activity of MAO-B is recorded on or in a medic alert article selected from a card, worn article, or radio frequency tag. In certain embodiments the mammal is human and the method further comprises informing the human of the assay result and/or its implications for the occurrence and/or progression of Parkinson's disease. In certain embodiments the method further comprises prescribing, or initiating and/or altering prophylaxis and/or therapy for Parkinson's disease in the subject when the determining provides a positive result. In certain embodiments the method further comprises scheduling the same test or a different test in a differential diagnostic protocol for Parkinson's disease. In certain embodiments the method further comprises scheduling follow-up diagnostics to monitor the subject for the onset of Parkinson's disease. In various embodiments the MAO-B nucleic acid and/or an MAO-B protein (or fragment thereof) is detected in an assay where the MAO-B protein or nucleic acid becomes labeled with a detectable label. In various embodiments the MAO-B nucleic acid and/or an MAO-B protein (or fragment thereof) is detected in an assay where the MAO-B protein or nucleic acid is transformed from a free state to a bound state by forming a complex with another assay component. In certain embodiments the MAO-B nucleic acid and/or an MAO-B protein (or fragment thereof) is detected in an assay where a MAO-B nucleic acid and/or a MAO-B protein or fragment thereof initially present in a soluble phase becomes immobilized on a solid phase. In various embodiments the MAO-B nucleic acid and/or an MAO-B protein or fragment thereof is detected in an assay where the sample is fractionated to separate MAO-B protein or nucleic acid from at least one other sample component. In certain embodiments the MAO-B nucleic acid and/or MAO-B protein or fragment thereof is detected in an assay where MAO-B nucleic acid or protein becomes embedded in a separation medium. In various embodiments the MAO-B nucleic acid and/or MAO-B protein or fragment thereof is detected in an assay where a MAO-B nucleic acid or protein is volatilized. In various embodiments the determining comprises determining activity of MAO-B in the sample. In certain embodiments the determining comprises reacting MAO-B in the sample with substrate for MAO-B (e.g., a monamine substrate, a benzylamine substrate, a phenylethylamine substrate, a fluorogenic substrate, a PET substrate etc.). In certain embodiments the determining comprises reacting MAO-B in the subject with a PET substrate and performing a PET scan on the subject.

In certain embodiments the determining comprises determining MAO-B (protein) expression level (e.g., transcription level) by determining for example the level of an MAO-B mRNA or in the sample. In certain embodiments the determining comprises transforming the sample or fraction derived therefrom by amplifying a reverse transcript of MAO-B mRNA from the sample. In certain embodiments the amplifying is by polymerase chain reaction (PCR) (e.g., reverse transcriptase PCR(RT-PCR), and/or quantitative PCR (Q-PCR), and the like). In certain embodiments the determining the expression level of an MAO-B nucleic acid comprises transforming mRNA in the sample or derived from the sample, by hybridizing mRNA in the sample or a reverse transcript derived therefrom to a probe that specifically hybridizes to a MAO-B nucleic acid. In certain embodiments the hybridizing is according to a method selected from the group consisting of a Northern blot, a Southern blot using DNA derived from a MAO-B RNA, an array hybridization, an affinity chromatography, and an in situ hybridization. In various embodiments the hybridizing comprises immobilizing the mRNA on a substrate by hybridizing the mRNA to a probe attached to a substrate. In various embodiments the hybridizing comprises fractionating the mRNA in a chromatography plate or system. In certain embodiments the probe is a member of a plurality of probes that forms an array of probes.

In certain embodiments determining the expression level of MAO-B comprises determining the amount of a MAO-B protein in the sample. In certain embodiments the determining is via a method selected from the group consisting of capillary electrophoresis, a Western blot, mass spectroscopy, ELISA, immunochromatography, and immunohistochemistry. In certain embodiments the determining comprises fractionating the MAO-B protein or a fragment thereof in a chromatography system or plate, or channel. In certain embodiments the determining comprises volatilizing the MAO-B protein or fragment thereof in a spectrometry system. In certain embodiments the spectrometry system comprises a mass spectrometer. In certain embodiments the determining comprises binding the MAO-B protein or fragment thereof with an anti-MAO-B antibody. In certain embodiments the determining comprises immobilizing the MAO-B protein or fragment thereof using the antibody.

In various embodiments methods of prophylactically treating a subject for Parkinson's disease are provided. The methods typically involve identifying a subject whose level of expression or activity of monoamine oxidase B (MAO-B) is elevated as compared to a control indicating that the mammal has an increased likelihood of developing Parkinson's disease; and prescribing for the subject and/or administering to the subject one or more MOA-B inhibitors. In various embodiments the subject is asymptomatic for Parkinson's disease. In various embodiments the subject presents with no significant neuronal cell loss (e.g., at the 15%, 10%, 5%, or 1% or better confidence level). In certain embodiments the subject is a human. In certain embodiments the subject is a human (e.g., infant, child, adolescent, or adult) that presents without symptoms of Parkinson's disease, but is believed to be at risk for the disease. In certain embodiments the subject is an infant or child. In various embodiments the samples, controls, and/or detection methods, etc. include, but are not limited to any of the methods described above or below herein (e.g., as illustrated in the claims).

In certain embodiments, this invention also pertains to the development of an animal model for Parkinson's disease and the use of that model for screening for drugs that inhibit MAO-B expression and/or activity. In various embodiments the animal model comprises a transgenic non-human mammal in which monoamine oxygenase B can be inducibly increased in astrocytes in the brain of the mammal. In certain embodiments the mammal is a rodent (e.g., a rat or mouse). In certain embodiments the mammal is a lagomorph. In certain embodiments the where astrocytes of the mammal comprise a nucleic acid encoding human MAO-B under the control of an inducible promoter. In certain embodiments astrocytes of the mammal comprise a nucleic acid encoding MAO-B under control of the bidirectional Tet-inducible promoter.

Methods of identifying an MAO-B inhibitor are also provided. In various embodiments the methods typically involve administering to an animal model (e.g. an animal model comprising a transgenic non-human mammal in which monoamine oxygenase B can be inducibly increased in astrocytes in the brain of the mammal, e.g., as described herein), one or more test agents; and detecting the expression and/or activity of MAO-B in the test animal under conditions where the MAO-B transgene is expected to be expressed and/or active, where a reduction in expression and/or activity of MAO-B indicates that the test agent(s) are inhibitors of MAO-B.

Also provided are kits for diagnosing a predisposition to Parkinson's disease. In certain embodiments the kits comprise a container containing a reagent and/or means for detecting MAO-B expression and/or activity; and instructional materials teaching the use of MAO-B expression and/or activity level(s) as a measure of risk for Parkinson's disease.

### DEFINITIONS

The terms Monoamine oxidase B (MAO-B)” as used herein refers to a polypeptide encoded by the MAOA-B gene, or naturally occurring variants thereof (see, e.g., Genbank NM_{—}000898, and the like) and/or fragments thereof that exhibit the spectrum of activity understood in the art for MAO-B.

The term “indicator” when used with respect to a prognostic assay (i.e., when elevated MAO-B expression or activity is said to be an indicator an increased likelihood of developing Parkinson's disease) need not require that the measured factor be dispositive of the presence or absence of the disorder or dispositive of the future occurrence of the disorder. The factor can simply indicate a predisposition to the disorder (e.g., a greater likelihood of presence or future occurrence of the disorder than is found in the absence of the indicator). It will be appreciated that such an indicator can often be one of a number of indicators used, typically in a differential diagnosis for the disease/disorder.

The term “MAO-B inhibitor” according to this invention or metabolite thereof, as used herein includes agents that inhibit the expression and/or activity of MAO-B in vivo. MAO-B inhibitors include pharmaceutical formulations of the active agent(s), including, for example, the “native” form or, if desired, in the form of salts, esters, amides, prodrugs, derivatives, and the like. Pharmaceutically acceptable salts of MAO-B inhibitors useful according to the methods of this invention include salts prepared from pharmaceutically acceptable reagents. In one embodiment, said pharmaceutically acceptable salt is a hydrochloride salt.

An “MAO-B” protein refers to a full-length MAO-B protein or to a fragment of an MAO-B protein that of sufficient length that it is recognizable as a fragment of the full length MAO-B and is indicative of the amount of MAO-B protein in a subject or sample, or that is derived by manipulation (e.g., in an assay) from a full-length MAO-B protein.

An “MAO-B” nucleic acid refers to a full-length MAO-B mRNA, a cDNA or other nucleic acid derived therefrom, wherein the amount of cDNA or other nucleic acid is indicative of the amount of MAO-B RNA transcribed in a subject or sample derived from a subject.

The term “medic alert” refers to information regarding a subjects health risks and/or health condition. In various embodiments the medic alert typically includes membership ID number, and/or primary medical condition(s), and/or a 24-Hour Emergency Response Center phone number.

The term “worn article” when used with reference to a MEDICALERT® refers to an article that is typically worn by a person and that contains a notification regarding one or more medical conditions of the person. Typical worn articles include, but are not limited to including but not limited to a watch, a bracelet, a pendant, a stretch band, a sports band, and the like.

The term “differential diagnosis” refers to the process by which a physician produces a diagnosis that explains a patient's symptoms. Typically, this process involves determining, from a set of possible candidates, which disease is causing the symptoms.

The term “medical record” or “patient medical record” refers to an account of a patient's examination and/or treatment that typically includes one or more of the following: the patient's medical history and complaints, the physician's physical findings, the results of diagnostic tests and procedures, and patient medications and therapeutic procedures. A “medical record” is typically made by one or more physicians and is written, transcribed or otherwise recorded record and/or history of various illnesses or injuries requiring medical care, and/or inoculations, and/or allergies, and/or treatments, and/or prognosis, and/or frequently health information about parents, siblings, and/or occupation. The record may be reviewed by a physician in diagnosing the condition.

### BRIEF DESCRIPTION OF THE DRAWINGS

FIG. 1, panels A-F, shows the characterization of transgenic mice that inducibly express human MAO-B specifically within astrocytes. Panel A: Schematic of transgenic constructs utilized to create transgenic mice with inducible elevation of human MAO-B (h-MAO-B) expression specifically within astrocytes via the bacterial tetracycline regulatory system. A bacterial reverse tetracycline response gene (rtetR) was expressed constitutively via a mouse glial fibrillary acidic protein promoter (pGFAP). The resulting gene product, rtTA, a tetresponsive transactivator protein, is activated only in the presence of doxycycline (dox) and is able to bind to a tetracycline operator (TET) sequence in the promoter of a second transgene, resulting in induction of expression of both h-MAO-B and bacterial betagalactosidase (lac Z) from the bidirectional promoter. Panel B: Human MAO-B cDNA transgene is expressed in the brains of transgenic mice following dox induction. Inducible transgene mRNA expression (a 448 by amplicon) was visualized by RT-PCR from RNA isolated from whole brains of uninduced (U), doxinduced (I) transgenic mice and wild type littermates (W). Panel C: Enzymatic activities of h-MAO-B and lacZ transgenes in whole brain lysates from uninduced (ND) and dox-induced (D) transgenic animals. n=3 animals per condition run in triplicate; * p<0.01. Data is presented as percent ND control. ND lacZ activity was calculated to be 11.9±0.54 nmoles ONPG converted/mg protein/hour and wild type C57BL6 lacZ activity (not shown) to be 11.3±0.13 nmoles ONPG converted/mg protein/hour respectively. Brain MAO-B activity in tissue isolated from the uninduced transgenic (ND) was calculated to be 14.6±0.9 nmoles 13-PEA converted/mg protein/hour and in tissues from wild type C57BL6 mice (not shown) to be 14.2±0.23 nmoles β-PEA converted/mg protein/hour. Panel D: Dox-inducible expression of human MAO-B (h-MAO-B) and lacZ is localized within GFAP⁺ astrocytes. GFAP immunostaining is shown in green, h-MAO-B is red, and merged in yellow in uninduced (No DOX) and dox-induced (DOX) animals. Scale bar applies to all images. Panel E: Loss in striatal dopamine (ST DA) content is exacerbated in dox-induced transgenics following a single MPTP injection of 30 mg/kg body weight. Striatal dopamine was estimated from wild type (W), uninduced transgenics (ND), induced transgenics (D), and dox-induced transgenics co-treated with deprenyl (Ddep). Grey bars without MPTP, black bars after a single injection of 30 mg/kg MPTP. Data is expressed as ng dopamine/mg striatal protein. * p<0.01 D vs. ND, **=p<0.01 Ddep vs. ND. Panel F: Loss in dopaminergic tyrosine-hydroxylase-positive (TH⁺) neurons in the substantia nigra (SN) after a single injection of MPTP (30 mg/kg) is exacerbated in dox-induced mice. Uninduced transgenic mice (ND), induced transgenics (Dox), and induced transgenics co-treated with 10 mg/kg deprenyl (Doxdep). Data is presented as percent of non-MPTP treated ND controls where SN TH⁺ cell numbers are estimated at 13,318±475 TH⁺ neurons in untreated ND mice (WT values, not shown, were 13,699±325). n=3 animals per condition run in triplicate; * p<0.05.

FIGS. 2A and 2B show inducible elevation of astrocytic MAO-B levels in mesencephalic cultures isolated from transgenic mice results in TH⁺ cell loss which is prevented by co-treatment with deprenyl. FIG. 2A: TH⁺ cell counts in uninduced mesencephalic cultures isolated from either wildtype littermates (WT), uninduced transgenics (ND) or transgenic cultures induced with 40 μg/ml doxycycline for 24 hrs in the absence (D) or presence of 10⁻6M deprenyl (Ddep). Counts are expressed as % WT values which were estimated to be 300±15 TH⁺ neurons. Cell counts were performed in a minimum of three different wells and 4 separate fields in each well. *=p<0.01 compared to ND; **p>0.01 compared to Dox. FIG. 2B: Representative micrographs of the TH+ cells obtained by TH+ immunofluorescence in uninduced (No Dox), dox-induced (Dox) and dox-induced mesencephalic cultures treated with deprenyl (Doxdep). Note the truncated processes and shrunken dopaminergic cell morphology in the induced cultures versus uninduced and deprenyl-treated induced cultures.

FIGS. 3A-3F show inducible elevation in astrocytic MAO-B results in selective, age-related SN dopaminergic neurodegeneration and global astrocyte activation even in the absence of neurotoxin. FIG. 3A: TH⁺ SN cell counts from uninduced transgenics (no dox) versus induced transgenics at 3-4 months (dox), following dox removal (dox rem) at 14 months of age (dox 14 mo), and induced co-treated with deprenyl at 10-30 mg/kg (dox dep 10-30), the anti-inflammatory agent minocycline at 10 mg/kg (dox mino) or the antioxidant Euk189 at 10 mg/kg in young animals. Data is expressed as total number of SN TH⁺ cells numbers per animal; *p<0.01 compared to ND, ** p<0.05 compared to Dox, and *** p<0.05 compared to dox. FIG. 3B: Neuronal (NeuN⁺) counts from uninduced (No Dox, filled bars) versus doxinduced transgenics (Dox, open bars) performed in equivalent nigral (SN), cortical (CT), and striatal (ST) tissue sections. Values represent average cell numbers per field in three separate fields per section, three sections per animal. N=5 animals per group. FIG. 3C: Representative micrographs demonstrating cortical astrocytic activation in doxinduced (Dox) versus uninduced (ND) animals and dox-induced animals co-treated with deprenyl (Ddep). Astrocytes from induced transgenics displayed enhanced GFAP staining and increased branching indicative of astrocyte activation (higher magnification in lower middle panel); deprenyl treatment resulted in reduced branching similar to that observed in uninduced controls (higher magnification in lower right-hand panel). FIG. 3D: Astrocytic (GFAP⁺) counts from uninduced (No DOX, open bars) versus doxinduced transgenics (DOX, light grey bars) and mice dox-induced in the presence of deprenyl co-treatment (D DEP, dark grey bars) performed in equivalent striatal (ST), nigral (SN), and cortical (CT) tissue sections. Values represent average cell numbers per field at 40× magnification in three separate fields per section, three sections per animal. N=5 animals per group; *p>0.05, ** p<0.1. FIG. 3E: Representative micrograph demonstrating increased expression of the astrocytic activation marker S-100â (blue) in GFAP cells in the transgenic cortex following dox induction. GFAP-labeled astrocytes (red) in induced animals display increased S-100â staining (pink) versus uninduced. FIG. 3F: Neurodegeneration of TH⁺ cells as assessed by silver staining. SN sections from uninduced (No Dox) and induced (Dox) animals were processed for T11⁺ immunoreactivity visualized by alkaline peroxidase (blue) and further processed for silver staining. A black punctate staining was observed in and around dying T11⁺ neurons (arrows) in Dox but not No Dox fields.

FIG. 4 shows that elevation in astrocytic MAO-B results in increased extracellular hydrogen peroxide (H₂O₂). Extracellular H₂O₂ concentrations were measured in media isolated from uninduced (ND) versus dox-induced (Dox) mesencephalic cultures in the absence and presence of 10 μM deprenyl (Dox Dep), 30 μM EUK189 (Dox Euk) or 500 nM apocynin (Dox Apo) using the Amplex Red assay. H₂O₂ concentrations were quantified against a standard H₂O₂ curve. Measurements were made on three separate media aliquots harvested prior to immunostaining of cultures; *p<0.01 compared to ND, ** p<0.05 compared to ND.

FIGS. 5A-5F show effects of elevations of astrocytic MAO-B on H₂O₂ and dopaminochrome (DAChr) levels, and mitochondrial complex I versus IV activities in isolated ST dopaminergic versus non-dopaminergic synaptosomes. FIG. 5A: Schematic of novel immunomagnetic technique utilized to for isolate ST dopaminergic versus non-dopaminergic synaptosomes. Synaptosomes were prepared from freshly dissected striata and the dopaminergic population rapidly isolated via immunoprecipitation with anti-DAT antibody following by separation by a magnetic particle-conjugated secondary antibody running through a strong magnetic column. Elution of the DA synaptosomes was obtained via removal of the magnetic field. Flow-through contains striatal non-dopaminergic synaptosomes used as negative controls in subsequent biochemical experiments. FIG. 5B: Densitometric quantitation of western blot analyses of fractions from immunomagnetic isolation of ST synaptosomes probed with TH, GABA or SNAP-25 antibodies. Levels of Dopaminergic TH (grey bars) versus Non-dopaminergic GABA (white bars) synaptosomes were assessed using SNAP-25, a general synaptic protein, as a loading control. Control (C), unbound fraction (U), flow-through (FT), wash (W) and eluate (E). Representative blots are shown in the lower panel. FIG. 5C: Elevation in astrocytic MAO-B results in increased _{H2O2} within dopaminergic ST synaptosomes. H₂O₂ levels were estimated 3 hrs following tail vein injection of DCFDA into either wildtype littermates (WT), uninduced (ND) or dox-induced (Dox) transgenics, or induced transgenic animals co-treated with either deprenyl (D DEP), EUK1 89 (D EUK) or 1 mg/ml catalase (Dox Cat). DCF fluorescence was examined at an excitation wavelength of 488 nm and emission at 512 nm. Data is reported normalized to microgram synaptosomal protein. N=3; *p<0.005 versus ND, **=p<0.01 versus Dox. FIG. 5D: Elevation in astrocytic MAO-B results in increased ST DAChr levels. DAChr levels were estimated in ST samples from uninduced (ND) versus dox-induced (Dox) transgenic mice via HPLC. Values are presented as picomoles DAChr/mg protein.* p<0.02, n=3. FIGS. 5E and 5F: Elevation in astrocytic MAO-B results in decreased complex I (FIG. 5E) but not complex IV (FIG. 5F) activity in ST dopaminergic synaptosomes. Complex I (FIG. 5E) and complex IV (FIG. 5F) activities were measured in isolated ST dopaminergic versus non-dopaminergic synaptosomes from uninduced (ND), dox-induced (Dox) and induced transgenic animals co-treated with either deprenyl (Dep) or EUK-189 (EUK). N=3-5 animals per group and a total of 3 separate experiments, values are expressed as mean±SD; * p<0.01 compared to ND, ** p>0.05 compared to ND. CI activity was calculated to be 4.0 versus 3.3 μM NADH/min/mg protein in ND striatal dopaminergic and non-dopaminergic synaptosomes, respectively. CIV activity was calculated to be 139 versus 109 μM ferrocytochrome c/minute/mg protein in ND striatal dopaminergic and non-dopaminergic synaptosomes, respectively. Activities of WT DA striatal synaptosomes was calculated to be 4.4 μM NADH/min/mg protein (CI) and 130 μM ferrocytochrome c/minute/mg protein (CIV); dox feeding of the WT animals did not alter the activities. Note: addition of doxycycline has been reported to interfere with mitochondrial protein translation including CI thereby affecting enzyme activity [62], however in addition to lack of effect on CI activity in WT animals+/−dox, we also find no change in CI subunit protein levels in WT mice as a consequence of dox treatment as assessed via immunoprecipitation/gel electrophoretic analyses (data not shown).

FIG. 6 shows that elevation in astrocytic MAO-B results in increased mitochondrial superoxide levels within dopaminergic SN mitochondria. Representative confocal micrographs illustrating increased merged (yellow) mitosox red fluorescence (red) in the SN and striatum of dopaminergic neurons following tail vein injection, fixation and TH immunochemistry (green) in uninduced (No Dox) versus induced transgenic mice (Dox) which is prevented in the presence of codeprenyl (DEP) or EUK-1 89 (EUK) treatment. Nuclei are visualized via DAPI staining (blue).

FIGS. 7A-7D show that global elevation in astrocytic MAO-B results in local SN microglial activation. FIG. 7A: Mitosox staining of local SN microglia following dox treatment reveals increased mitochondrial superoxide levels within these cells. Microglia were immunostained with Iba1 (green) antibody in sections of brain fixed 3 hrs following tail injection of mitosox red. Confocal detail shows mitochondrial mitosox red fluorescence within an activated microglial cell body following dox induction. FIG. 7B: Microglial activation occurs following dox induction within the SN. Microglial activation as assessed by Iba1 immunochemistry (black, DAB-nickel staining) in SN sections from uninduced (No Dox), induced (Dox), and dox-induced codeprenyl-treated transgenics (Ddep). Dopaminergic (TH⁺) neurons are labeled in brown (DAB). Note that few activated microglia are visible in No Dox and Ddep treated mice versus in the Dox panel where several stages of microglial activation (resting, activated, withdrawing, and motile stages) can be visualized. Scale bar equals 50 μM unless indicated otherwise. FIG. 7C: Higher magnification of representative SN Iba1 immunochemistry demonstrating stages of microglial activation (activated, withdrawing, and motile stages are indicated by cartoons). Cortical microglia were found to be in a ramified or resting stage in all the conditions (FIG. 10). FIG. 7D: Representative Iba1 immunochemistry in ST sections demonstrating increased microglial activation following dox induction. Shown are ST sections from uninduced (No Dox) versus dox-induced (Dox) or dox-induced, co-deprenyl treated (Ddep) transgenics. Note the presence of numerous retracted processes in the microglia in the Dox panel versus presence of ramified microglia in the No Dox and Ddep panels.

FIGS. 8A and 8B show elevation in astrocytic MAO-B results in loss of ambulatory movement in MAO-B transgenics. Total ambulatory velocity (FIG. 8A) and ambulatory distance (FIG. 8B) following two weeks of dox administration. Data is presented as average ambulatory velocity calculated over a 10 minute observation period and total ambulatory distance is for that same time interval. N=5 mice per condition; experiments were run in triplicate. * p<0.01 compared to ND, ** p>0.05 compared to ND. Values are given for untreated (ND), dox-treated (Dox), and dox, deprenyl co-treated mice. Wildtype untreated littermates had an ambulatory velocity of 2.37±0.45 cm/s and a total ambulatory distance moved of 1082±101 cm.

FIG. 9 shows a schematic representation of the events occurring upon the elevation of glial MAO-B. Increased astroglial levels of MAO-B results in elevation of H₂O₂ levels; this can be prevented by deprenyl. Astroglia are themselves protected from H₂O₂ due to relatively high levels of glutathione-gluathione peroxidase versus neurons. H₂O₂ can diffuse out of astroglia and into neighboring cells including local dopaminergic neurons and microglia; this can be prevented by EUK. Dopamine can be oxidized by H₂O₂ through dopamine quinone formation to stable dopaminochrome (DAC11R). DAC11R can extract an electron from the auto-oxidizable site of mitochondrial complex I forming DACR radical and impacting selectively on complex I function. Due its high affinity for molecular oxygen, DACR radical can pass an electron on to this molecular species resulting in its re-reduction to DAC11R and increased formation of mitochondrial superoxide radical. MAO-B generated H₂O₂ can also stimulate local microglia; microglial activation can be greatly secondarily enhanced within the SN due to dopaminergic cell demise which can also exacerbate ROS production and impact on local dopaminergic neurons.

FIG. 10 shows that microglial activation is largely absent in the cortex. Absence of detectable microglial activation in the cortical sections from untreated, dox-treated deprenyl cotreated animals. Iba1 immunocytochemistry reveals that cortical microglia are in a resting ramified stage in all the three types of treatments in contrast to what occurs in the striatum and the substantia nigra (FIG. 6). This suggests that activation in the nigrostriatum may largely be due to secondary effects as a consequence of SN dopaminergic demise. Two representative micrographs are provided from each treatment.

### DETAILED DESCRIPTION

In order to specifically assess the role of MAO-B in Parkinson's Disease (PD), transgenic mice were created in which MAO-B can be inducibly increased in astrocytes, the normal brain cell population in which it is expressed. Experiments peformed on these mice indicated that elevation of MAO-B results in a specific, selective and progressive loss of midbrain dopaminergic neurons accompanied by selective inhibition of mitochondrial complex I, increased mitochondrial oxidative stress, local microglial activation, and locomotor dysfunction; all hallmarks of human Parkinson's disease. These transgenic animals constitute a novel model system for exploring pathways involved in initiation and progression of key features of Parkinson's disease pathology and for therapeutic drug testing. More specifically, data based on these animal studies shows that relative MAO-B elevation is a novel early biomarker predictive of later disease development and that MAO-B inhibition is warranted in those individuals displaying increased levels of the enzyme upon assay.

Accordingly in certain embodiments, methods are provided for identifying a mammal at risk for Parkinson's disease. The methods typically involve determining the level of expression and/or activity of monoamine oxidase B (MAO-B) in the mammal and/or in a sample from the mammal where an elevated level of MAO-B expression and/or activity as compared to a control (e.g., a threshold valued determined for a population) is an indicator that the mammal has an increased likelihood of developing Parkinson's disease. The MAO-B activity or expression levels can be determined according to any of a number of methods known to those of skill in the art. Typically the sample is a sample of a biological fluid or tissue in which MAO-B activity and/or expression levels are reflective of (e.g., correlated with) MAO-B expression and/or activity levels in the brain. Suitable samples include, but are not limited to saliva, blood, blood fractions, cerebrospinal fluid, and the like. In certain embodiments, the MAO-B activity level is determined in vivo, e.g., using a PET scan.

It will be recognized that, in certain embodiments, th diagnostic assays described herein can be incorporated as components in a differential prognostic diagnosis for Parkinson's disease (PD). When performed, for example, in a clinical setting (e.g., hospital, doctor's office etc.), the methods can further involve recording the determined level of MAO-B expression or activity, and/or a diagnosis based at least in part on the determined level of MAO-B expression or activity, in a patient medical record. Typically medical records include, but are not limited to a hospital medical record, a doctor's office medical record, an insurance company medical record, a health maintenance organization (hmo) medical record, a personal medical record, a laboratory medical record, a personal medical record website, a computer readable medium storing a medical record, and a radio frequency tag (RF tag). In certain embodiments in patients identified as “at risk” for Parkinson's disease, the diagnosis, based at least in part on the level of expression or activity of MAO-B is recorded on or in medical alert (e.g., a MEDICALERT®) article such as a card, a worn article, or radio frequency tag. Typically where the methods are diagnostic/prognostic performed in a clinical setting, the methods can further involve informing the tested subject of the assay result and/or its implications for the occurrence and/or progression of Parkinson's disease. In certain embodiments the method further comprises prescribing, or initiating and/or altering prophylaxis and/or therapy for Parkinson's disease the subject when the assay provides a positive result. Typically, where there is a positive assay result (e.g., a prognosis of PD), the methods can further involve scheduling follow-up diagnostics to monitor said subject for the onset of Parkinson's disease.

Prophylactic treatment methods are also provided. For example, a subject can be evaluated for elevated MAO-B expression and/or activity. A positive assay, indicating the subject is at risk for developing Parkinson's disease can then be an indicator for prophylactic therapy, e.g., administration of one or more MAO-B inhibitors to prevent or delay the onset and/or severity of the disease.

Also provided is an animal model of Parkinson's disease. The animal model comprises a transgenic animal (e.g., a transgenic rodent) that inducibly expresses MAO-B (e.g., a heterologous MAO-B). The creation of such a transgenic mouse is illustrated herein in the Examples. Using the methods described herein, other transgenic rodent models are readily prepared. The model animal(s) can be used to screen various test agents for MAO-B inhibitory activity.

I. Assays for MAO-B Expression and/or Activity.

In various embodiments, the diagnostic/prognostic methods as well as the prophylactic therapeutic methods and the drug screening methods described herein involve measuring the expression and/or activity of MAO-B. Numerous assays for measuring MAO-B activity as well as assays for determining MAO-B expression levels are known to those of skill in the art.

A) MAO-B Activity Assays.

Monoamine oxidase (MAO) is widely distributed throughout the body and catalyzes the oxidative deamination of a variety of monoamines. MAO has been classified into two main types: MAO-A and MAO-B. MAO-A deaminates serotonin (5-HT) and noradrenaline (NA) much better than phenylethylamine (PEA) or benzylamine, and is preferentially inhibited by clorgyline, whereas, MAO-B prefers PEA and benzylamine as substrates and is preferentially inhibited by 1-deprenyl.

Monoamine oxidase B (MAO-B) activity can be measured by any of a number of methods. Methods of measuring MAO-B are well known to those of skill in the art and such methods include, but are not limited to (A) direct biochemical tests, including, but not limited to: 1) radiometry, in platelets; 2) radiometry, in tissue samples; 3) gas chromatography-mass spectrometry (GC-MS); 4) spectrophotometry; 5) luminometry 6) high throughput fluorescence assay; 7) high performance liquid chromatography (HPLC); 8) Berthelot Reaction; 9) Immunoblotting; 10) Radiolabelling; 11) Positron emission tomography; or (B) indirect biochemical tests, including, but not limited to: 1) Urine Concentrations By GC-MS; 2) HVA Concentrations in Urine Samples; 3) Urine and Plasma Concentrations of PEA and related substances; 4) Plasma PEA Concentrations by GC-MS; 5) Urine Concentrations of PEA & Related Substances by GC-MS; 6) Urine concentrations of HVA by GC; or 7) HVA In Cerebro-Spinal Fluid (CSF) by GC-MS; or (C) by genetic tests in any tissue sample of fluid, including, but not limited to blood, saliva, cerebrospinal fluid, and the like.

In certain embodiments, MAO-B activity is measured in platelets. MAO-B activity can be measured in platelets as described, for example, by Wurtman and Axelrod (1963) Biochem Pharmacol. 12: 1417-1419), Jackman et al. (1979) Clin Chim Acta. 96(1-2): 15-23), Shekim et al. (1984) Psychiatry Res. 11(2): 99-106), Young et al. (1986) Arch Gen Psychiatry 43(6):604-609), Hallman et al. (1987) Acta Psychiatr. Scand. 76(3): 225-34); Garpenstrand et al. (2000) J. Neural Transm. 107(5): 523-530; Whitfield et al. (2000) Psychol Med. 30(2): 443-454, and the like.

In certain embodiments, the biological activity of MAO-B can be measured as described by Flaherty et al. (1996) J. Med. Chem., 39: 4756-4761, using 1-methyl-4-(1-methyl-2-pyrryl)-1,2,3,6-tetrahydropyridine as a substrate. This is a one-step fluorometric method for the measurement of monoamine oxidase (MAO) activity in 96-well microplates with high sensitivity. This assay is based on the detection of H₂O₂ in a horseradish peroxidase-coupled reaction using N-acetyl-3,7-dihydroxyphenoxazine (Amplex Red), a highly sensitive and stable probe for H₂O₂. With a single sampling, this assay is useful for performing both end-point and continuous measurements of MAO activity. Using a commercially available enzyme, the assay allows the detection of MAO B activity as low as 1.2×10⁻⁵ U/ml.

In certain embodiments, MAO-B activity in PRP is measured by the method described by McEwen (1971) Meth. Enzymol., Colowick, S. P. and Kaplan, N, O. eds, pp. 692-693. Academic Press, New York). This approach utilizes a spectrophotometric assay based on the measurement of the conversion of benzylamine into benzaldehyde by the catalytic activity of MAO-B.

In certain embodiments, MAO-B activity can be detected using PET activity assays. Thus, for example, subjects can be administered intravenous L-¹¹C] deprenyl tracer and scanned using positron emission tomography (PET). A dose of 20 mg of L-deprenyl can readily be used to assay MAO-B activity (see, e.g., Bench et al. (1990) Eur. J. Clin. Pharmacol., 40(2): 169-173, and the like).

B) Nucleic-Acid Based Assays.

In certain embodiments, in addition to, or as an alternative to measuring MAO-B activity, MAO-B expression levels are determined. This typically involves measuring MAO-B mRNA levels (e.g., MAO-B transcription levels) and/or MAO-B protein levels.

1) Target Molecules.

Changes in MAO-B expression level can be detected by measuring changes in MAO-B mRNA levels and/or a nucleic acid derived from the MAO-B mRNA (e.g. reverse-transcribed cDNA, expressed protein, etc.). In order to measure MAO-B transcription and/or expression level it is desirable to provide a nucleic acid sample for such analysis. In certain embodiments the nucleic acid is found in or derived from a biological sample. The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes.

It was a surprising discovery that nucleic acids derived from tissues other than neurological tissues (e.g., from blood cells) can provide effective diagnostic and/or prognostic indicators of a psychiatric disorder or a predilection to such a disorder. Thus, in certain embodiments, the biological sample is a sample comprising cells of neurological origin and/or non-neurological origin. In certain embodiments, the biological sample comprises blood cells (e.g., peripheral blood lymphocytes and/or lymphoblastic cell lines).

The nucleic acid (e.g., MAO-B mRNA, or a nucleic acid derived from MAO-B mRNA) is, in certain embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In certain embodiments the “total” nucleic acid is isolated from a given sample using, for example, an acid guanidinium-phenol-chloroform extraction method and polyA+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see. e.g, Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA_{—}86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level the nucleic acid sample is one in which the concentration of the MAO-B nucleic acid is proportional to the transcription level (and therefore expression level) of that gene. Similarly, in hybridization assays, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required, appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript, or large differences or changes in nucleic acid concentration are desired, no elaborate control or calibration is required.

In the simplest embodiment, the nucleic acid sample is the total mRNA or a total cDNA isolated and/or otherwise derived from a biological sample (e.g., a sample from a neural cell or tissue, a peripheral blood cell, etc.). The nucleic acid can be isolated from the sample according to any of a number of methods well known to those of skill in the art as indicated above.

2) Hybridization-Based Assays.

Using the known sequence of MAO-B (see, e.g., Genbank NM_{—}000898), detecting and/or quantifying the transcript(s) (e.g., MAO-B mRNA) or other MAO-B nucleic acids (e.g., cDNA reverse transcribed from MAO-B mRNA) can be routinely accomplished using nucleic acid hybridization techniques (see, e.g., Sambrook et al. supra). For example, one method for evaluating the presence, absence, or quantity of a reverse-transcribed cDNA involves a “Southern Blot”. In a Southern Blot, the DNA (e.g., reverse-transcribed MAO-B mRNA), typically fragmented and separated on an electrophoretic gel, is hybridized to a probe specific for that nucleic acid (e.g., a probe complementary to part or all of the MAO-B nucleic acid). Comparison of the intensity of the hybridization signal from the target specific probe with a “control” probe (e.g. a probe for a “housekeeping gene) provides an estimate of the relative expression level of the target nucleic acid (e.g, MAO-B mRNA).

Alternatively, in certain embodiments the MAO-B RNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes can be used to identify and/or quantify the target mRNA. Appropriate controls (e.g. probes to housekeeping genes) can provide a reference for evaluating relative expression level.

An alternative means for determining the MAO-B expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use can vary depending on the particular application.

In some applications it is necessary to block the hybridization capacity of repetitive sequences. Thus, in some embodiments, tRNA, human genomic DNA, or Cot-1 DNA is used to block non- specific hybridization.

3) Amplification-Based Assays.

In another embodiment, amplification-based assays can be used to measure MAO-B expression (transcription) level. In such amplification-based assays, the target nucleic acid sequences (e.g., MAO-B nucleic acid(s)) act as template(s) in amplification reaction(s) (e.g. Polymerase Chain Reaction (PCR), reverse-transcription PCR (RT-PCR), etc.). In a quantitative amplification, the amount of amplification product will be proportional to the amount of template in the original sample. Comparison to appropriate controls (e.g., similar measurements made for samples from healthy mammals) provides a measure of the transcript level.

Methods of “quantitative” amplification are well known to those of skill in the art. For example, in certain embodiments, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.).

One illustrative internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The nucleic acid sequence(s) provided herein are sufficient to enable one of skill to routinely select primers to amplify any portion of the gene(s).

In certain embodiments, the MAO-B expression levels can be determined using a real-time PCR assay. Real-time polymerase chain reaction, also called quantitative real time polymerase chain reaction (QRT-PCR) or kinetic polymerase chain reaction, is a technique based on polymerase chain reaction, which is used to amplify and simultaneously quantify a targeted DNA molecule. It enables both detection and quantification (as absolute number of copies or relative amount when normalized to DNA input or additional normalizing genes) of a specific sequence in a DNA sample. The procedure follows the general principle of polymerase chain reaction; its key feature is that the amplified DNA is quantified as it accumulates in the reaction in real time after each amplification cycle. Two common methods of quantification are the use of fluorescent dyes that intercalate with double-strand DNA, and modified DNA oligonucleotide probes that fluoresce when hybridized with a complementary DNA. Frequently, real-time polymerase chain reaction is combined with reverse transcription polymerase chain reaction to quantify low abundance messenger RNA (mRNA), enabling a researcher to quantify relative gene expression at a particular time, or in a particular cell or tissue type.

Real-time PCR using double-stranded DNA dyes involves the use of a DNA-binding dye (e.g., SYBR Green) that binds to all double-stranded (ds)DNA in a PCR reaction, causing fluorescence of the dye. An increase in DNA product during PCR therefore leads to an increase in fluorescence intensity and is measured at each cycle, thus allowing DNA concentrations to be quantified. However, dsDNA dyes such as SYBR green bind to all dsDNA PCR products, including nonspecific PCR products (“primer dimers”). This can potentially interfere with or prevent accurate quantification of the intended target sequence. When using DNA-binding dyes, the PCR reaction is typically prepared as usual, with the addition of the fluorescent dsDNA dye. The reaction is run in a thermocycler, and after each cycle, the levels of fluorescence are measured with a detector; the dye only fluoresces when bound to the dsDNA (i.e., the PCR product). With reference to a standard dilution, the dsDNA concentration in the PCR can be determined.

Like other real-time PCR methods, the values obtained do not have absolute units associated with it (i.e. mRNA copies/cell). A comparison of a measured DNA/RNA sample to a standard dilution gives a fraction or ratio of the sample relative to the standard, allowing relative comparisons between different tissues, samples, or experimental conditions. To ensure accuracy in the quantification, it is usually necessary to normalize expression of a target gene to a stably expressed gene (see below). This can correct possible differences in RNA quantity or quality across experimental samples.

Using fluorescent reporter probes is the most accurate and most reliable of the methods. This approach uses a sequence-specific RNA or DNA-based probe (e.g., a probe complementary to the MAO-B amplification product) to quantify only the DNA containing the probe sequence. Use of the reporter probe thus significantly increases specificity, and allows quantification even in the presence of some non-specific DNA amplification. Reporter probe real-time PCR methods are commonly carried out with an RNA- or DNA-probe with a fluorescent reporter at one end and a quencher of fluorescence at the opposite end of the probe. The close proximity of the reporter to the quencher prevents detection of its fluorescence; breakdown of the probe by the 5′ to 3′ exonuclease activity of the taq polymerase breaks the reporter-quencher proximity and thus allows unquenched emission of fluorescence, which can be detected. An increase in the product targeted by the reporter probe at each PCR cycle therefore causes a proportional increase in fluorescence due to the breakdown of the probe and release of the reporter. When using fluorescent probes, the PCR reaction is typically prepared as usual, and the reporter probe is added. As the reaction commences, during the annealing stage of the PCR both probe and primers anneal to the DNA target. Polymerisation of a new DNA strand is initiated from the primers, and once the polymerase reaches the probe, its 5′-3-exonuclease degrades the probe, physically separating the fluorescent reporter from the quencher, resulting in an increase in fluorescence. Fluorescence is detected and measured in the real-time PCR thermocycler, and its geometric increase corresponding to exponential increase of the product is used to determine the threshold cycle (CT) in each reaction.

In one approach to quantifying real-time PCR, relative concentrations of DNA present during the exponential phase of the reaction are determined by plotting fluorescence against cycle number on a logarithmic scale (so an exponentially increasing quantity will give a straight line). A threshold for detection of fluorescence above background is determined. The cycle at which the fluorescence from a sample crosses the threshold is called the cycle threshold, Ct. Since the quantity of DNA doubles every cycle during the exponential phase, relative amounts of DNA can be calculated, e.g. a sample whose Ct is 3 cycles earlier than another's has 2³=8 times more template.

Amounts of RNA or DNA can then be determined by comparing the results to a standard curve produced by RT-PCR of serial dilutions (e.g. undiluted, 1:4, 1:16, 1:64) of a known amount of RNA or DNA. As mentioned above, to accurately quantify gene expression, the measured amount of RNA from the gene of interest is divided by the amount of RNA from a housekeeping gene measured in the same sample to normalize for possible variation in the amount and quality of RNA between different samples. This normalization permits accurate comparison of expression of the gene of interest between different samples, provided that the expression of the reference (housekeeping) gene used in the normalization is very similar across all the samples. Methods of performing quantitative real-time PCR are well known to those of skill in the art (see, e.g. Dorak (2006) Real Time PCR (BIOS Advanced Methods), Taylor & Francis, New York; Edwards (2004) Real-Time PCR: An Essential Guide, Taylor & Francis, New York; King and O'Connell (2002) RT-PCR Protocols (Methods in Molecular Biology), Humana Press, Totowa, N.J., and the like).

4) Hybridization Formats and Optimization of Hybridization

i. Array-Based Hybridization Formats.

In certain embodiments, the methods of this invention can be utilized in array-based hybridization formats. Arrays typically comprise a multiplicity of different “probe” or “target” nucleic acids (or other compounds) attached to one or more surfaces (e.g., solid, membrane, or gel). In certain embodiments, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211, Nuber (2005) DNA Microarrays (Bios Advanced Methods), Taylor & Francis, New York, N.Y., and the like.)

Arrays, particularly nucleic acid arrays, can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

The simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934.

ii. Other Hybridization Formats.

A variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P labelled probes or the like. Other labels include ligands that bind to labeled antibodies, fluorophores, chemi-luminescent agents, enzymes, and antibodies that can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

5) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results, and that provides a signal intensity greater than approximately 2 times, preferably greater than approximately 4 times and more preferably greater than approximately 10 times the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in Tijssen (1993) Laboratory Techniques in Biochemistry and Molecular Biology, Vol. 24: Hybridization With Nucleic Acid Probes, Elsevier, N.Y.)

Methods of optimizing hybridization conditions are well known to those of skill in the art (see, e.g., Tijssen (1993) supra).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

6) Labeling and Detection of Nucleic Acids.

The probes used herein for detection of MAO-B nucleic acids can be full length or less than the full length of the mRNA(s) encoding the particular component(s) of interest. Shorter probes are empirically tested for specificity. Preferred probes are sufficiently long so as to specifically hybridize with the target nucleic acid(s) under stringent conditions. The preferred size range is from about 10, about 15, or about 20 bases to the full length of the encoding mRNA, more preferably from about 30 bases to the length of the mRNA, and most preferably from about 40 bases to the length of mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵ I, ³⁵S, ¹⁴C or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

In certain embodiments a fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

Desirably, fluorescent labels should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label can be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

C) Polypeptide-Based Assays.

In certain embodiments MAO-B transcription levels can be determined by measuring the amount of MAO-B protein present in the sample. MAO-B proteins can be detected and quantified by any of a number of methods well known to those of skill in the art. These can include, but are not limited to analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In certain embodiments, MAO-B polypeptide(s) are detected/quantified in an electrophoretic protein separation (e.g., a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In certain embodiments, Western blot (immunoblot) analysis is used to detect and quantify the presence of the MAO-B polypeptide(s) in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) can be directly labeled or alternatively may be subsequently detected using labeled antibodies (e.g., labeled sheep anti-mouse antibodies) that specifically bind to the a domain of the antibody.

In certain embodiments, the MAO-B polypeptide(s) are detected using an immunoassay. As used herein, an immunoassay is an assay that utilizes an antibody to specifically bind to the analyte (e.g., the target MAO-B polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (e.g., MAO-B polypeptide). In certain embodiments, the capture agent is or comprises an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G can also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

In certain embodiments preferred immunoassays for detecting the MAO-B polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide (e.g., MAO-B polypeptide) thus immobilized is then bound by a labeling agent, such as a second antibody bearing a label.

In competitive assays, the amount of analyte (e.g. MAO-B protein) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one embodiment, the antibody is immobilized on a solid substrate. The amount of target polypeptide bound to the antibody may be determined either by measuring the amount of target polypeptide present in a polypeptide/antibody complex, or alternatively by measuring the amount of remaining uncomplexed polypeptide.

In various embodiments the immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind a MAO-B polypeptide, either alone or in combination. In the case where the antibody that binds the polypeptide is not labeled, a different detectable marker, for example, an enzyme-labeled antibody capable of binding to the monoclonal antibody which binds the MAO-B polypeptide, can be employed. Any of the known modifications of EIA, for example, enzyme-linked immunoabsorbent assay (ELISA), may also be employed. As indicated above, also contemplated by the present invention are immunoblotting immunoassay techniques such as Western blotting employing an enzymatic detection system.

In various embodiments the immunoassay methods of the present invention can also include other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds the MAO-B polypeptide is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents can also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, are commercially available or can be produced using standard methods well know to those of skill in the art.

It will also be recognized that antibodies can be prepared by any of a number of commercial services (e.g., Berkeley antibody laboratories, Bethyl Laboratories, Anawa, Eurogenetec, etc.).

The foregoing assay methods are intended to be illustrative and not limiting. Using the teachings provided herein other assay formats for evaluating MAO-B transcript level will be recognized by one of skill in the art.

### II. Treatment Methods.

In certain embodiments, this invention contemplates methods of prophylactic treatment of Parkinson's disease to prevent or slow the onset and/or progression of the disease. The methods typically involve identifying a subject (e.g., an apparently asymptomatic human) who tests positive in an assay for elevated MAO-B activity. When, particularly in the context of a differential diagnosis, the subject is classified as having elevated risk or susceptibility for Parkinson's disease, a prophylactic amount of one or more MAO-B inhibitor(s) are prescribed/administered to the subject to slow or prevent the onset of the disease and/or to reduce its severity.

MAO-B Inhibitors.

In certain embodiments, the MAO-B inhibitor(s) include, but are not limited to selegiline (JUMEX®, JUMEXAL® CARBEX®, ELDEPRYL®, MOVERGAN®; APTAPRYL®, ANIPRYL®; ELDEPRINE®; PLURIMEN®), desmethylselegiline, pargyline (EUDATIN®, SUPIRDYL®, EUTONYL®, see, e.g., U.S. Pat. No. 3,155,584, which is incorporated herein by reference), rasagiline [R(+)N-propargyl-laminoindan], 3-N-phenylacetylamino-2,5-piperidinedione, caroxyazone, AGN-1135 (see, e.g., WO 92/21333), MDL 72195 (see, e.g., WO 92/21333), J 508 (see, e.g., WO 92/21333), lazabemide (see, e.g., WO 00/45846), milacemide (see, e.g., WO 00/45846), IFO (see, e.g., WO 00/45846], mofegiline (see, e.g., WO 00/45846), and 5-(4-(4,4,4-trifluorobut-oxy)phenyl)-3-(2-methoxyethyl)-1,3,4-oxadiazol-2(3H)-one (see, e.g., WO 00/45846).

Other various known monoamine oxidase inhibitors include, Isocarboxazid (MARPLAN®), Moclobemide (AURORIX®, MANERIX®, MOCLODURA®), Phenelzine (NARDIL®), Tranylcypromine (PARNATE®, JATROSOM®), Nialamide, Iproniazid (MARSILID®, IPROZID®, IPRONID®, RIVIVOL®, PROPILNIAZIDA®), Methylene Blue, Turmeric (active ingredient curcumin), harmaline, cigarettes (the active compound has not yet been identified, but is a strong inhibitor on MAO-B, with no action on MAO-A), Iproclozide, Toloxatone, Linezolid (ZYVOX®, ZYVOXID®). In addition, many tryptamines have MAOI properties. Harmine (present in Harmal, Banisteriopsis caapi, and tobacco) is a powerful MAOI. Certain synthetic tryptamines such as AMT or 5-MeO-AMT produce only minor MAO inhibition. In certain embodiments the use of these MAO inhibitors is contemplated. In certain embodiments one or more of the following are excluded: tumeric, methylene blue, harmaline, cigarettes (or active commpounds therein), antibiotics, and harmine.

In certain embodiments, prodrugs or metabolites of the MAOB inhibitors are contemplated. Typically the metabolite has substantially the same or better selective MAO-B inhibitor activity as its unmetabolized form.

In certain embodiments, a prodrug of a MAOB inhibitor comprises a derivatized MAOB inhibitor that is metabolized in vivo into the active inhibitory agent. Prodrugs typically have substantially the same or better therapeutic value than the underivatized MAOB inhibitor(s). For example, a prodrug useful according to this invention can improve the penetration of the drug across biological membranes leading to improved drug absorption; prolong duration of the action of the drug, e.g., slow release of the parent drug from the prodrug and/or decrease first-pass metabolism of the drug; target the drug action; improve aqueous solubility and stability of the drug (e.g., intravenous preparations, eyebrows etc.); improve topical drug delivery (e.g., dermal and ocular drug delivery); improve the chemical and/or enzymatic stability of drugs (e.g., peptides); or decrease side effects due to the drug. Methods for making prodrugs are readily known in the art.

Pharmaceutical Formulations.

In order to carry out the methods of the invention, one or more active agents e.g., MAO-B inhibitors) are administered to a mammal in need thereof, e.g., to a mammal having a increased likelihood of developing Parkinson's disease. In various embodiments the mammal is asymptomatic for Parkinson's disease when the MAO-B inhibitors are prescribed/administered.

The active agent(s) (one or more MAO-B inhibitors) can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, the disulfide salts of a number of delivery agents are described in PCT Publication WO 00/059863 which is incorporated herein by reference. Similarly, acid salts of therapeutic peptides, peptoids, mimetics, and other agents and can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. Certain preferred basic salts include alkali metal salts, e.g., the sodium salt, and copper salts.

Preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the active agents (e.g., MAO-B inhibitors described herein) are useful for parenteral, topical, oral, nasal (or otherwise inhaled), rectal, or local administration, such as by aerosol or transdermally, typically for prophylactic use to inhibit the onset and/or severity of Parkinson's disease. The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, lipid complexes, etc.

The active agents (e.g., MAO-B inhibitors) can also be combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., MAO-B inhibitor) and the resulting composition is compressed. Where necessary the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In therapeutic applications, the compositions of this invention are administered, e.g., topically administered or administered to the oral or nasal cavity, to a patient suffering from infection or at risk for infection or prophylactically to prevent dental caries or other pathologies of the teeth or oral mucosa characterized by microbial infection in an amount sufficient to prevent and/or cure and/or at least partially prevent or arrest the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms in) the patient.

The concentration of active agent(s) can vary widely, and will be selected primarily based on activity of the active ingredient(s), body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic and/or phophylactic regimen in a particular subject or group of subjects.

In certain embodiments, the active agents of this invention are administered to the oral cavity. This is readily accomplished by the use of lozenges, aerosol sprays, mouthwash, coated swabs, and the like.

In certain embodiments, the active agent(s) of this invention are administered topically, e.g., to the skin surface, to a topical lesion or wound, to a surgical site, and the like.

In certain embodiments the active agents of this invention are administered systemically (e.g., orally, or as an injectable) in accordance with standard methods well known to those of skill in the art. In other preferred embodiments, the agents, can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Other formulations for topical delivery include, but are not limited to, ointments, gels, sprays, fluids, and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

As indicated above, various buccal, and sublingual formulations are also contemplated.

In certain embodiments, one or more active agents of the present invention can be provided as a “concentrate”, e.g., in a storage container (e.g., in a premeasured volume) ready for dilution, or in a soluble capsule ready for addition to a volume of water, alcohol, hydrogen peroxide, or other diluent.

While the invention is described with respect to use in humans, it is also suitable for animal, e.g., veterinary use. Thus certain preferred organisms include, but are not limited to humans, non-human primates, canines, equines, felines, porcines, ungulates, lagomorphs, and the like.

It is also noted that, as indicated above, a number of MAO-B inhibitors are commercially available pharmaceuticals and appropriate/tolerable dosage ranges have been determined.

The foregoing formulations and administration methods are intended to be illustrative and not limiting. It will be appreciated that, using the teaching provided herein, other suitable formulations and modes of administration can be readily devised.

### III. Parkinson's Disease Model Animals.

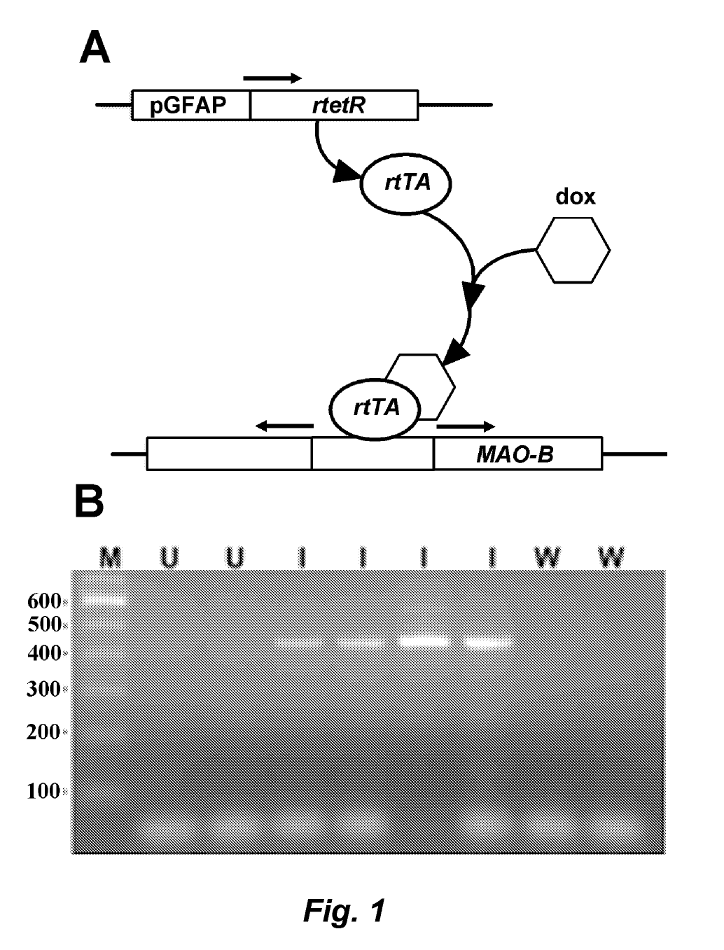

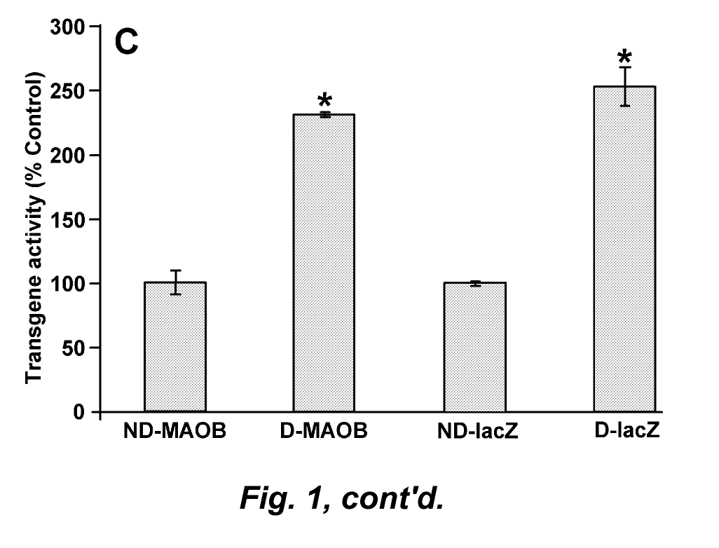

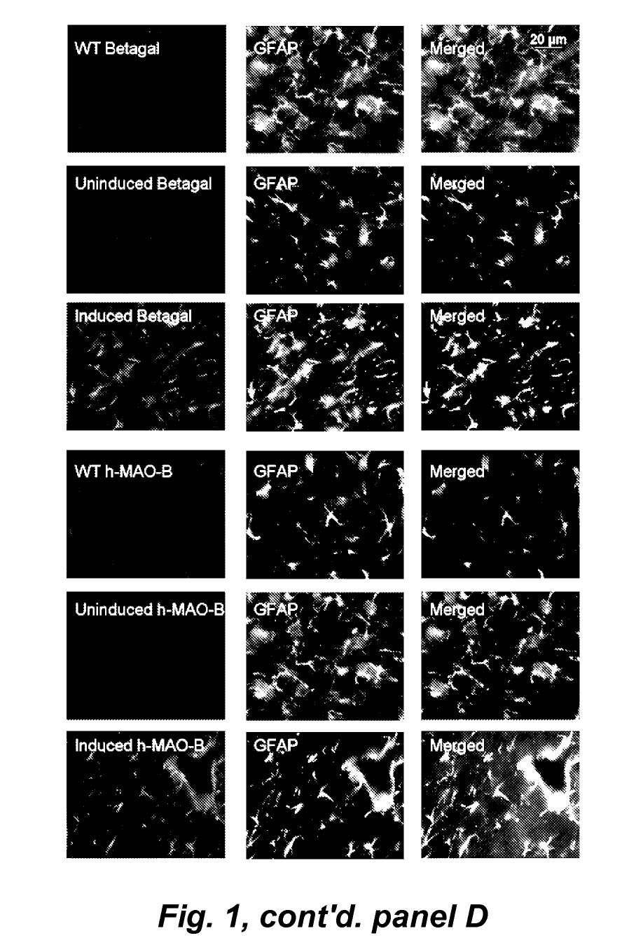

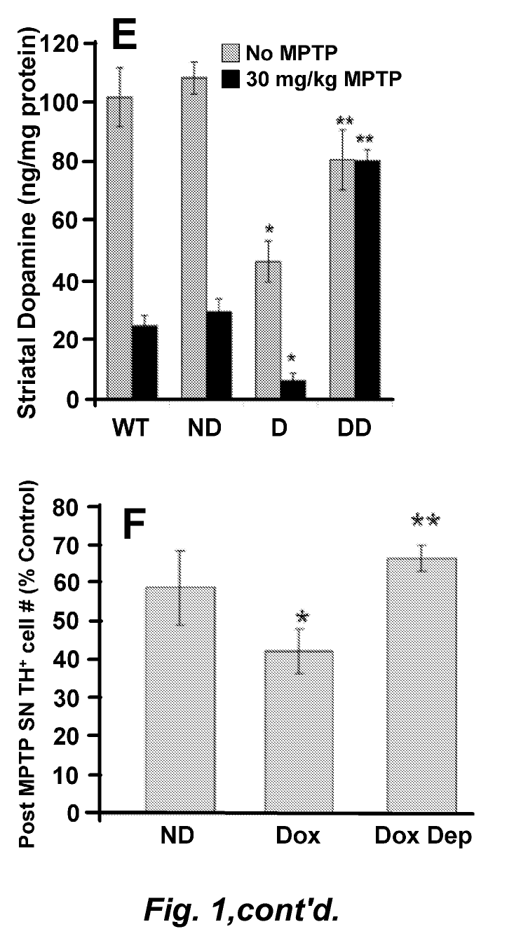

In certain embodiments, this invention pertains to the creation of transgenic animal models for Parkinson's disease. The animal models comprise a non-human mammal that inducible increases MAO-B in astrocytes, the normal brain cell population in which MAO-B. In certain embodiments the mammal is a rodent (e.g., a mouse that constitutively expresses a reverse tetracycline responsive transactivator (rtTa) protein specifically within astrocytes via a glial fibrillary acidic protein promoter (pGFAP)). Upon addition of the tetracycline derivative doxycycline (dox), rtTa binds to a tetracycline responsive bidirectional promoter (TET) and induces simultaneous expression of both wildtype human MAO-B cDNA (GenBank accession number NM000898) and, an optional marker protein bacterial beta-galactosidase (lacZ) (see, e.g., FIG. 1, panel A). Expression of the two co-transgenes could be induced via inclusion of dox the animal's food.

Results from our studies suggest that elevation of MAO-B results in a specific, selective and progressive loss of midbrain dopaminergic neurons accompanied by selective inhibition of mitochondrial complex I, increased mitochondrial oxidative stress, local microglial activation, and locomotor dysfunction all hallmarks of the human condition. These novel transgenics constitute a novel mouse model system for generally exploring pathways involved in initiation and progression of key features of pd pathology and for therapeutic drug testing.

While a transgenic mouse is illustrated in the Examples, having demonstrated that such a transgenic mammal is viable, other transgenic species (e.g., transgenic rodents), can readily be prepared.

### IV. Screening for MAO-B Inhibitors.

In certain embodiments, methods are provided for screening for inhibitors of MAO-B expression and/or activity. The methods typically involve administering one or more test agents to a transgenic animal that can inducibly express a heterologous MAO-B. The animal is tested for MAO-B activity under conditions in which MAO-B expression and/or activity is expected to be elevated (e.g., where the heterologous MAO-B is induced), and the animal is assayed for elevated MAO-B expression and/or activity and/or for one of the symptoms of Parkinson's disease, e.g., selective inhibition of mitochondrial complex I, increased mitochondrial oxidative stress, local microglial activation, locomotor dysfunction, etc. If MAO-B expression and/or activity and/or one or more of the symptoms is reduced, the test agent is identified as a candidate MAO-B inhibitor and/or a potential agent for prophylactic and/or therapeutic treatment of Parkinson's disease.

### V. Kits.

In certain embodiments this invention provides kits for the practice of the methods of this invention. The kits typically comprise a reagent or reagents for assaying the activity or transcript level of a MAO-B nucleic acid in a sample. Thus, for example, the kits can comprise primers for amplifying a MAO-B nucleic acid, and/or probes that specifically hybridize to a nucleic acid, and/or immunoassay reagent(s) for detecting MAO-B peptide (e.g., an anti-MAO-B peptide antibody), and/or substrates for measuring MAO-B activity, and the like.

The kits can optionally include, materials, devices or reagents for performing the methods described herein. Thus, for example, the kits can optionally comprise labels, devices for obtaining a biological sample, devices for storing a biological sample, reagents for extracting proteins and/or nucleic acids, and the like.

In addition, the kits optionally include labeling and/or instructional materials providing directions (i.e., protocols) for the practice of the methods of this invention. Preferred instructional materials describe assays for MAO-B expression and/or activity as a prognostic indicator (e.g., in a differential diagnosis) for the presence of or elevated risk for Parkinson's disease.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### MAO-B Elevation in Mouse Brain Astrocytes Results in Parkinson's Pathology

Age-related increases in monoamine oxidase B (MAO-B) may contribute to neurodegeneration associated with Parkinson's disease (PD). The MAO-B inhibitor deprenyl, a long-standing antiparkinsonian therapy, is currently used clinically in concert with the dopamine precursor L-DOPA. Clinical studies suggesting that deprenyl treatment alone is not protective against PD associated mortality were targeted to symptomatic patients. However, dopamine loss is at least 60% by the time PD is symptomatically detectable, therefore lack of effect of MAO-B inhibition in these patients does not negate a role for MAO-B in pre-symptomatic dopaminergic loss. In order to directly evaluate the role of age-related elevations in astroglial MAO-B in the early initiation or progression of PD, we created genetically engineered transgenic mice in which MAO-B levels could be specifically induced within astroglia in adult animals. Elevated astrocytic MAO-B mimicking age related increase resulted in specific, selective and progressive loss of dopaminergic neurons in the substantia nigra (SN), the same subset of neurons primarily impacted in the human condition. This was accompanied by other PD-related alterations including selective decreases in mitochondrial complex I activity and increased mitochondrial oxidative stress. Along with a global astrogliosis, we observed local microglial activation within the SN. These pathologies correlated with decreased locomotor activity. Importantly, these events occurred even in the absence of the PD-inducing neurotoxin MPTP. Our data demonstrates that elevation of murine astrocytic MAO-B by itself can induce several phenotypes of PD, indicating that MAO-B is directly involved in multiple aspects of disease neuropathology. Mechanistically this may involve increases in membrane permeant H₂O₂ which can oxidize dopamine within dopaminergic neurons to dopaminochrome which, via interaction with mitochondrial complex I, can result in increased mitochondrial superoxide. Our inducible astrocytic MAO-B transgenic provides a novel model for exploring pathways involved in initiation and progression of several key features associated with PD pathology and for therapeutic drug testing.

### Materials and Methods

Generation and Analysis of Transgenics with Inducible Astrocyte-Specific Elevation of MAO-B Levels

Human MAO-B cDNA was cloned into pBIG (Clontech) at the PstI and SalI sites, placing the gene under transcriptional control of the bidirectional Tet-inducible promoter. A second construct was prepared which contained the 3.7 kb HinDIII-BamHI fragment of the mouse GFAP promoter cloned in EcoRI blunt ended pTetOn vector, driving expression of the Tet-responsive reverse transactivator rtTA. Transgenic C57B16 mice inducibly expressing human MAO-B selectively within astrocytes were produced by co-injecting the two transgenes into the male pronuclei of a fertilized egg prior to implantation into a pseudopregnant female. Subsequent founder mice were crossed with wildtype C57B16 and resulting litters genotyped by PCR using the primers 5′-ACT GAA CCC AAA GGC ACA C-3′ (SEQ ID NO:1) and 5′-AAC ATG ACA ATG AAG GAG CTA C-3′ (SEQ ID NO:2) for MAO-B, and 5′-GCC TTT ACC CAT TAC C-3′ (SEQ ID NO:3) and 5′-CCC GCT TAT TTT TAA TGC-3′ (SEQ ID NO:4) for rTta. Littermates positive for both transgenes were mated to achieve homogeneous lines. Astroglial-specific transgene expression was induced by feeding animals doxycycline at 3000 ppm provided in pre-mixed Purina chow (Research Diets) for a three week period. Assuming a 5 g diet per day for a 30 g mouse, the dosage achieved was calculated to be equivalent to 0.5 g/kg/day. Total RNA was isolated from cortical brain tissue of wildtype uninduced and uninduced transgenic mice using Trizol (Invitrogen) according to manufacturer's instructions. cDNA was prepared using Superscript III reverse transcriptase kit (Invitrogen) and oligo-(dT) primers from 2 μg of total RNA. PCR was carried out with the forward and reverse primers corresponding to human MAO-B cDNA as above using Taq polymerase (Eppendorf). A 448 bp amplicon indicates the induction of the h-MAO-B transcript. Positive transgene induction was also confirmed based on MAO-B and betagalactosidase positive-immunostaining within astrocytes of dox-induced transgenics using antibodies specific to hMAO-B and betagalactosidase within GFAP⁺ cells. Betagalactosidase and MAO-B enzyme activities were also performed in whole brain homogenates from induced versus uninduced transgenic lines [2,55]. The line showing the highest level of induction was used for all subsequent studies. Mice were housed according to standard animal care protocols, fed ad libitum, kept on a 12 hr light/dark cycle, and maintained in a pathogen-free environment in the Buck Institute Vivarium. Animals used for subsequent studies were either young adults (2-6 months of age) or older animals (14 months of age) fed dox on the regime described above. Co-treatment with other agents involved intraperitoneal administrations of 10-30 mg/kg/day of deprenyl, minocycline or EUK-189 for two weeks.

MPP⁺ Measurements, Striatal Dopamine Content Cell Counts and Neurodegeneration in Inducible Astrocytic MAO-B Transgenics Versus Controls+/−MPTP.

Striatal tissue was harvested from dox versus untreated animals 90 minutes following injection of 30 mg/kg MPTP intraperitoneally, immediately immersed in ice cold 0.1 M perchloric acid and sonicated. MPP⁺ was measured by HPLC as previously described [56]. Dopamine levels were analyzed in striata dissected two weeks of induction or 24 hrs following MPTP injection post induction by HPLC followed by a 464 pulsed electrochemical detection (Neurochemistry Core, Center for Molecular Neuroscience, Vanderbilt University). Stereological cell counts were performed on immunostained brain sections from brains harvested two weeks after induction using either antibody against tyrosine hydroxylase (TH, Chemicon, 1:500) followed by biotin-labeled secondary antibody and development using DAB (Vector Laboratories). TH⁺ cells were counted stereologically throughout the SNpc [56]. Counts for dox removal were performed on brains dissected after removal of dox from the feed for a three week period. Sections were cut at a 40 μm thickness, and every 4th section was counted using a grid of 100×100 μm. Dissector size used was 35×35×12 μm. Neuronal numbers were assessed following NeuN⁺ (Chemicon, 1:100) or GABA⁺ (Chemicon, 1:1000) immunostaining and astrocytes via GFAP⁺ (DAKO, 1:500) and s100-â (DAKO, 1:200) immunostaining. Cells were counted in the SN, striata, and cortex in three independent sections per condition and ten fields per section. Neurodegeneration in the TH⁺ cells was visualized by silver staining (FD Neurotechnologies, Ellicott City, Md.) according to the manufacturer's instructions using proprietary compounds after immunostaining the sections with antibody against tyrosine hydroxylase (TH, Chemicon, 1:500) visualized with Vector Blue alkaline phosphatase (Vector labs). Dopaminochrome was measured from striatal samples by HPLC as per Ochs et al [39].

Extracellular H₂O₂ Levels and Dopaminergic Cell Survival in Mesencephalic Cultures

Primary mixed cultures were prepared from the midbrain of 14-day-old mice embryos from MAO-B transgenics and WT controls. Tissue was digested in Neurobasal medium containing 30 U/ml papain and 20 μg/ml DNase at 37° C. for 30 min and mechanically triturated. Dissociated cells were centrifuged at 500×g, resuspended in growth medium (Neurobasal medium supplemented with 10% FBS, 2 mM glutamate, B25 supplement without antioxidants, 50 U/ml penicillin, 50 U/ml streptomycin and 50 ng/ml GDNF [57]), and plated on poly-d-lysine-coated 8 well chamber slides (BD-Biocoat) at a density of 10⁵ cells per ml. Mixed cultures were grown at 37° C. for 3-5 days before induction with 40 μg/ml doxycycline for 12 hours as per [22]. Cells were co-treated with 10 μM deprenyl or 500 nM apocynin. H₂O₂ in the medium was assayed by Amplex Red per the manufacturer's instructions. Dopaminergic and microglial cell counts were performed following fixation of the cultured cells for 15 min with 4% formaldehyde following by T11⁺ (Chemicon) or Iba1⁺ (WAKO USA, Richmond, Va.) immunostaining or via uptake of ASP⁺ (4-(4-(dimethylamino)styryl)-N-methylpyridinium iodide) (Invitrogen), a fluorescent analogue of dopamine. 10 μM ASP⁺ was loaded on to the uninduced or induced cultures in the presence of 20 μM of desipramine to inhibit the norepinephrine transporter which would also take up the dye. ASP⁺ fluorescence (red) was visualized in the T11⁺ cells after immunostaining with anti-T11 antibody and Alexa fluor 488 conjugated secondary antibody. Averages of manual double blind T11⁺ or fluorescent counts of three wells of culture with the same treatment were collected; counts were performed in triplicate.

Immuno-Magnetic Isolation of Dopaminergic Synaptosomes

Dopaminergic and non-dopaminergic striatal synaptosomes were isolated using a modified immuno-magnetic protocol [58]. Briefly, synaptosomes were prepared from dissected striatal tissue [59]. Dopaminergic synaptosomes were isolated using magnetic beads conjugated to anti-rabbit IgG (Miltenyi Biotech) after capturing them using a rabbit anti-DAT antibody (Chemicon). Under a magnetic field, bead-bound synaptosomes were passed through a column and washed; flow-through in the presence of the magnetic field yielded a fraction enriched in non-dopaminergic striatal synaptosomes. Elution performed in the absence of the magnetic field yielded fractions enriched in dopaminergic striatal synaptosomes. Equal quantities of various fractions of the synaptosomes isolated using DAT antibody including the starting material or control, the unbound fraction, the flow-through, the wash and the eluate were subjected to western analysis to assess relative dopaminergic versus non-dopaminergic synaptosomal purification. Western blots were performed using antibodies against TH (Chemicon 1:500), GABA (Chemicon, 1:1000) or SNAP-25 as a synaptic protein normalization control (Chemicon, 1:1000). Band densities were quantified using a Chemimager (Alpha Innotech, San Leandro, Calif.). Approximately 94% synaptosomes in the eluate were dopaminergic according to densitometric evaluation of the western blot.

H₂O₂ Estimation in Synaptosomes

Hydrogen peroxide levels were measured in the striatal dopaminergic and nondopaminergic synaptosomes prepared as above from various groups of mice. Mice were injected in the tail vein with ˜200 μl of DCFDA (Calbiochem) which was freshly diluted 100× with PBS from a stock of 100 mg/ml in DMSO as modified from Andrews et. al. [38]. The animals were sacrificed 3 hours later and synaptosomes were prepared as above. Hydrogen peroxide in the synaptosomes with or without 1 mg/ml catalase for 30 min after preparation was visualized as DCF fluorescence at 488 nm excitation and 512 nm emission in a Spectramax Gemini fluorescence plate reader. The relative fluorescence was normalized to synaptosomal protein which was quantified using the Bradford reagent, Bio-Rad.

Mitochondrial Complex I and IV Activities

Isolated synaptosomes were found to be physiologically viable for up to 3 hours. Complex I activities were assayed in isolated dopaminergic and non-dopaminergic synaptosomal fractions from dox-induced animals as rotenone-sensitive NADH dehydrogenase activity by measuring DCPIP (2,6-dichlorophenolindophenol) reduction in synaptosomal extracts following addition of 200 μM NADH, 200 μM decylubiquinone, 2 mM KCN, and 0.002% DCPIP in the presence and absence of 2 μM rotenone [60]. Complex IV activity was assayed as cytochrome c oxidase activity by observing the rapid (1-2s) oxidation of freshly reduced 40 μM ferrocytochrome c in a 10 mM K-PO₄ buffer pH 7.2 containing 100 mM KCl, 0.025% maltoside at 30° C., at 550 nm, averaged over two concentrations per sample. Values for all assays were normalized/protein using BioRad reagent.

Detection of Mitochondrial Superoxide In Vivo

Mitosox Red (Molecular Probes) was used as an indicator to assess presence of superoxide within cellular mitochondria. 10 μg of Mitosox in 200 μl phosphate-buffered saline was injected into the tail vein of dox-induced transgenics versus controls. Animals were sacrificed 90 minutes later, brains rapidly dissected out and post-fixed overnight in 4% paraformaldehyde. Brains were sectioned at 30 μm using a cryostat. Cellular localization and site of Mitosox Red accumulation in the nigrostriatum was identified by immunofluorescence using anti-TH (Chemicon, 1:500), anti-IBA1 (Wako, 1:500) or anti-GFAP (DAKO, 1:500) antibodies visualized with Fluorescein Avidin D. Mitochondrial localization was confirmed by immunoanalysis using an anti-ATP synthase-specific antibody. Fluorescent imaging was done using a Zeiss LSM510 NLO confocal microscope and images procured using LSM software. Quantification of colocalization and fluorescent intensity was determined using the IMARIS software suite (Bitplane AG, Zurich, Switzerland) on selected cells from multiple sections.

Morphological Analyses of Astrocyte and Microglial Activation

Immunochemistry was performed on cryosections derived from the SN, striata, and CTX of fixed perfused brains from dox-induced transgenics versus controls. Astrocytic activation was assessed using primary antibodies against GFAP and s100-β. Microglial activation was detected using primary antibodies against that Horse radish peroxidase or avidin-conjugated secondary antibodies were used to detect primary antibodies following either DAB or Fluorescein/Texas Red staining.

Locomotor Behavioral Assessment

Locomotor function was assessed using an automated Truscan photobeam apparatus (Colbourne Instruments, Allentown, Pa.) under illumination as previously described [61]. Animals were habituated to the apparatus for 10 min then data collected over a 10-min period followed by analysis using Truscan 99 software. Spontaneous horizontal movement was assessed in both control and dox treated versus untreated transgenic mice via both ambulatory velocity and distance in a 10-min period.

### Results

Creation of Transgenic Mouse Lines in which Astrocytic MAO-B Activity can be Inducibly Increased in Adult Animals.

Double transgenic mouse lines were generated in our laboratory that constitutively express a reverse tetracycline responsive transactivator (rtTa) protein specifically within astrocytes via a glial fibrillary acidic protein promoter (pGFAP). Upon addition of the tetracycline derivative doxycycline (dox), rtTa binds to a tetracycline responsive bidirectional promoter (TET) and induces simultaneous expression of both wildtype human MAO-B cDNA (GenBank accession number NM000898) and the marker protein bacterial beta-galactosidase (lacZ) (FIG. 1, panel A). Expression of the two co-transgenes was induced in our studies via inclusion of dox in the normal mouse chow, and visualized by RT-PCR (FIG. 1, panel B). Whole brain extracts from induced animals with the highest levels of expression exhibited an approximate 2.5- fold increase in MAO-B and lacZ activities versus uninduced controls (FIG. 1, panel C); increased MAO-B activity was completely inhibited by deprenyl treatment (data not shown). This high-expressing line was used for all subsequent studies. Immunohistochemistry using antibodies specific to MAO-B demonstrated that the human MAO-B was exclusively expressed within (GFAP) astroglia throughout the brain and only following dox treatment (FIG. 1, panel D).

Elevation in Astrocytic MAO-B Results in Increased Conversion of MPTP to MPP⁺ and Significant Increases in Toxin-Induced Dopaminergic SN Cell Loss.

To validate that our transgenic model expresses functionally active MAO-B, we assessed its vulnerability to acute systemic MPTP administration. Since conversion of MPTP to MPP₊ is catalyzed by MAO-B, we predicted that elevation in functional astrocytic MAO-B should result in increased striatal MPP⁺ levels and exacerbation of selective MPTP-induced dopaminergic SN neurodegeneration. Induced and uninduced transgenic mice and wildtype C57B16 littermates were treated with 30 mg/kg MPTP and striatal MPP levels measured via HPLC (Table 1). At 90 minutes post MPTP injection, striatal MPP levels were 1.5-1.8-fold that observed in either uninduced transgenic or wildtype controls. This increase in striatal MPP⁺ was accompanied by a significant decrease in striatal dopamine levels and dopaminergic SN cell numbers in induced transgenics compared to controls seven days following MPTP treatment (FIG. 1, panels E & F). MAO-B elevation alone was found to result in ˜60% reduction in striatal dopamine levels (FIG. 1, panel E) which was exacerbated further by MPTP. Elevations in astrocytic MAO-B in our transgenics resulted in exacerbation of MPTP-induced neurotoxicity suggesting that astrocytic MAO-B increases had the expected functional effect.

TABLE 1
Striatal MPP+ levels.
MPP+ Levels

| | MPP+ |
|---|---|
| | (ng/mg protein) |
| Dox | 182 ± 3.4 |
| No Dox | 118 ± 2.3 |
| WT | 100 ± 3.5 |

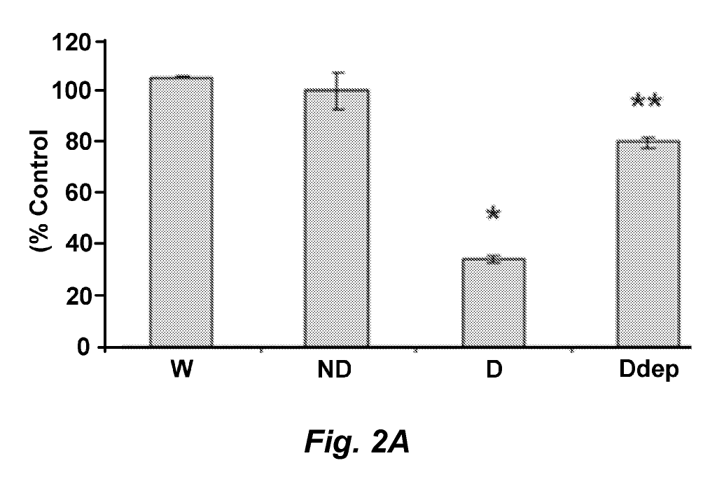

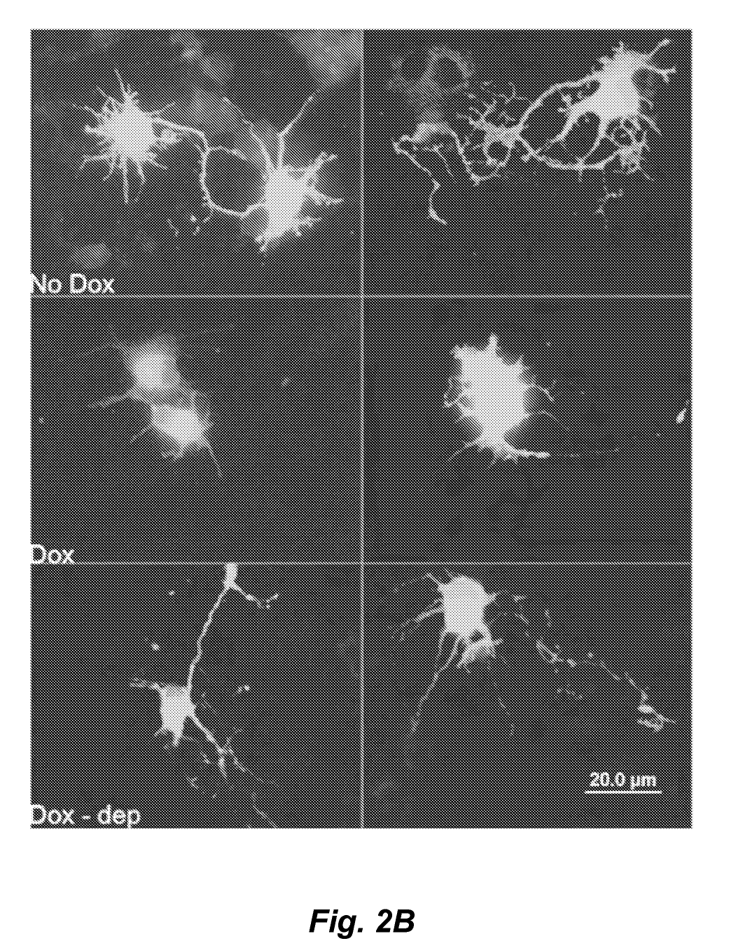

Astrocytic MAOB elevation in mesencephalic cultures isolated from inducible MAO-B lines was found to result in dopaminergic neurodegeneration. Mesencephalic cultures were prepared from day-14 embryos from transgenic mice and wildtype littermates. Mixed neuronal/glial mesencephalic cultures were grown on coated plates for 3 days and transgenic MAO-B activity induced in a portion of the transgenic cultures by addition of 40 μg/ml dox. Dox was added to wildtype cultures as a negative control. Twelve hours following dox addition, immunocytochemistry performed on dox-induced transgenic cultures revealed a significant loss in the number of tyrosine hydroxylase positive (TH⁺) cells compared to controls even in the absence of toxin addition that was attenuated by deprenyl co-treatment (FIG. 2A). To verify that loss of TH immunostaining represents actual preferential loss of dopaminergic cells, we counted the number of cells within the cultures which take up the fluorescent dopamine analogue ASP⁺ (4-(4-(dimethylamino)styryl)-N-methylpyridinium iodide, Molecular Probes) as a measure of cells with functional dopamine transporter activity. We found a similar percentage loss of ASP⁺ cells (˜80%, 44±22 versus 244±68 uninduced per field). This demonstrates that the loss in TH⁺ cell numbers is not simply a consequence of selective loss of this particular marker. In addition to increased dopaminergic cell loss, the remaining dopaminergic neurons were observed to undergo significant loss of their neurite processes which became stunted and shrunken in size. Deprenyl co-treatment attenuated not only the extensive dopaminergic cell loss observed following induction of MAO-B within cultured astrocytes but also the observed morphological dopaminergic neurite alterations (FIG. 2B).

Elevations of Astrocytic MAO-B In Vivo Results in Selective Dopaminergic SN Cell Loss.

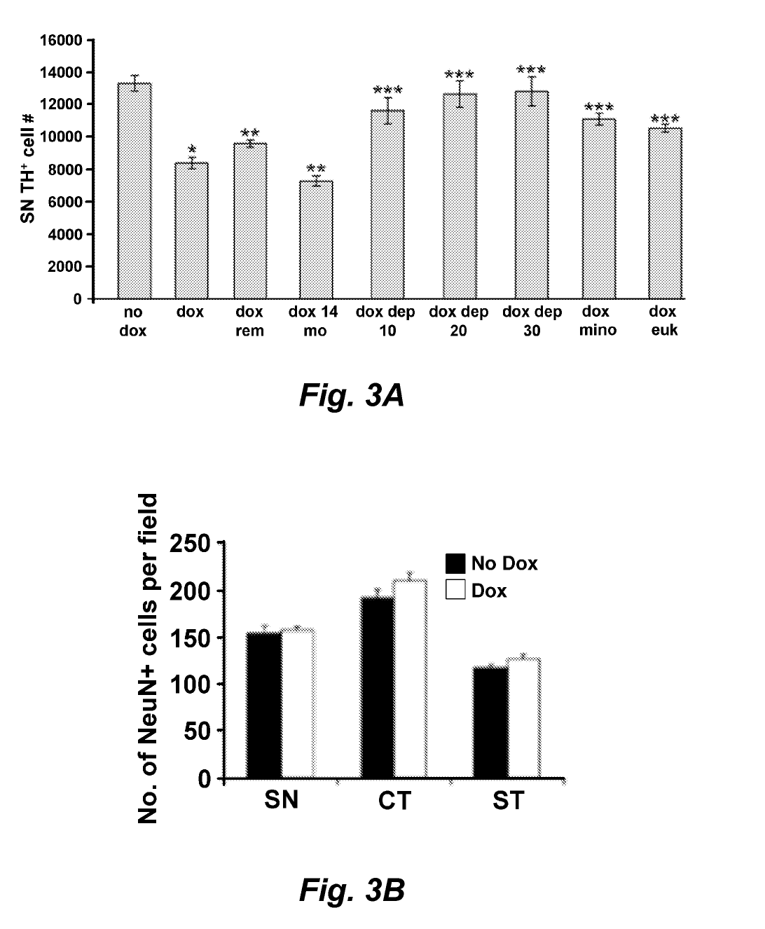

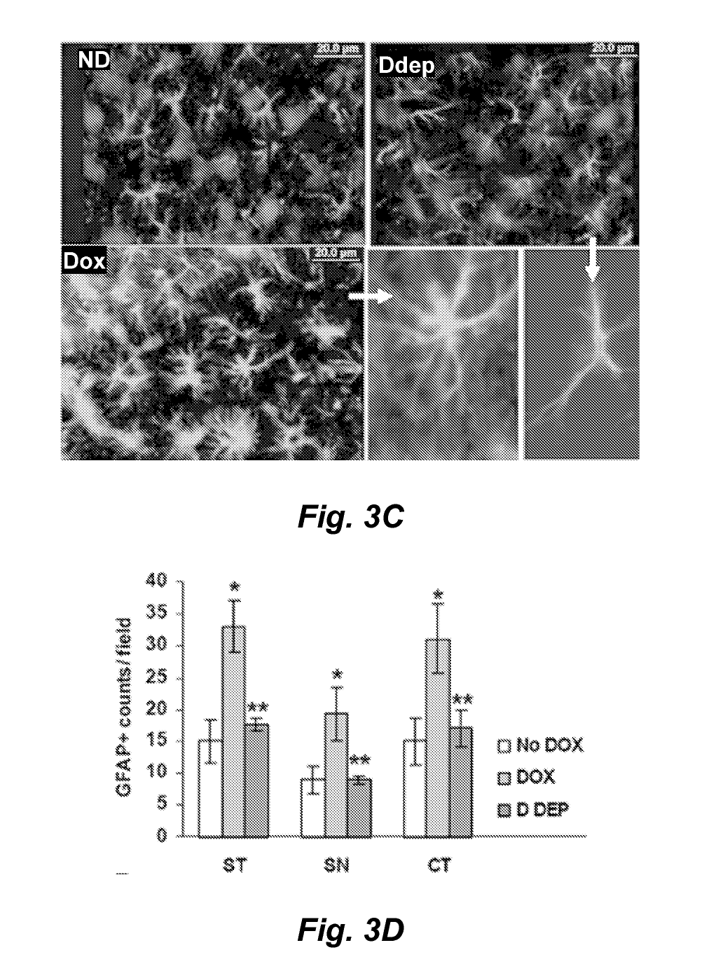

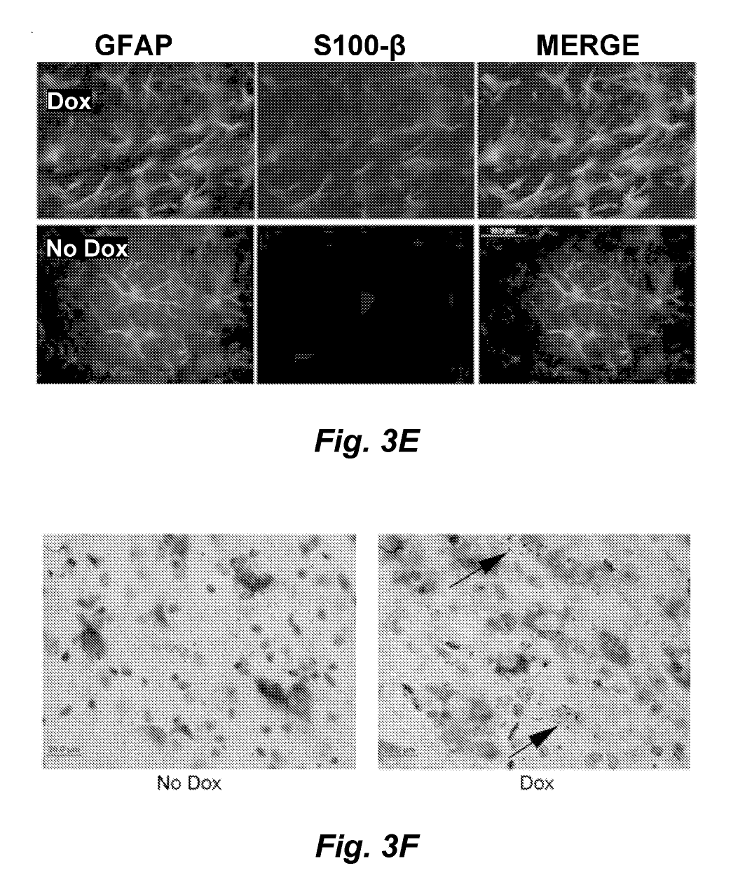

To assess the impact of astrocytic MAO-B elevation on its own in vivo on the nigrostriatal system, stereological dopaminergic cell counts were performed on dox-fed transgenic mice versus controls. A two week induction period in 2-3 month old transgenics resulted in an approximate 40% loss in dopaminergic SN cell numbers compared to either untreated transgenics or wildtype littermates; this loss was prevented by deprenyl co-treatment (FIG. 3 A). Moreover, removal of dox from the animal feed after the initial two weeks for three weeks did not result in a reversal of TH⁺ counts to normal levels. Astrocytic MAO-B elevation in older (14 mo.) transgenics resulted in an even more pronounced dopaminergic SN cell loss (˜50%) than that observed in the younger animals. In contrast, total neuronal numbers in the SN as assessed by NeuN staining did not differ between dox-treated transgenics versus untreated controls nor were neuronal cell numbers in the striatum or cortex impacted by astrocytic MAO-B elevation (FIG. 3B), even though it occurred throughout the brain and resulted in a general and widespread astrogliosis as evidenced by GFAP immunocytochemistry in these same brain regions (FIGS. 3C, 3D). GABAergic SN cell counts also revealed no difference in doxinduced versus non-induced animals further suggesting that the observed dopaminergic cell loss in this brain region was selective (data not shown). GFAP immunostaining revealed profuse branching as normally observed following astroglial activation and increased astroglial brain numbers following dox treatment which were inhibited by deprenyl. Levels of S100-β, a marker for activated astrocytes, were also increased in transgenic astroglia following dox induction (FIG. 3E).

We performed silver staining to verify that selective loss of TH′ immunostained cells in the SN following dox induction coincided with increased neurodegeneration in this brain region (FIG. 3F). This data along with the dox removal results suggests that the decrease in TH⁺ SN cells is not merely due to a reversible selective loss of the TH⁺ marker but constitutes an irreversible dopaminergic SN neurodegeneration.

Astrocytic MAO-B Elevations Result in Increased Mitochondrial Oxidative Stress in Dopaminergic SN Neurons.

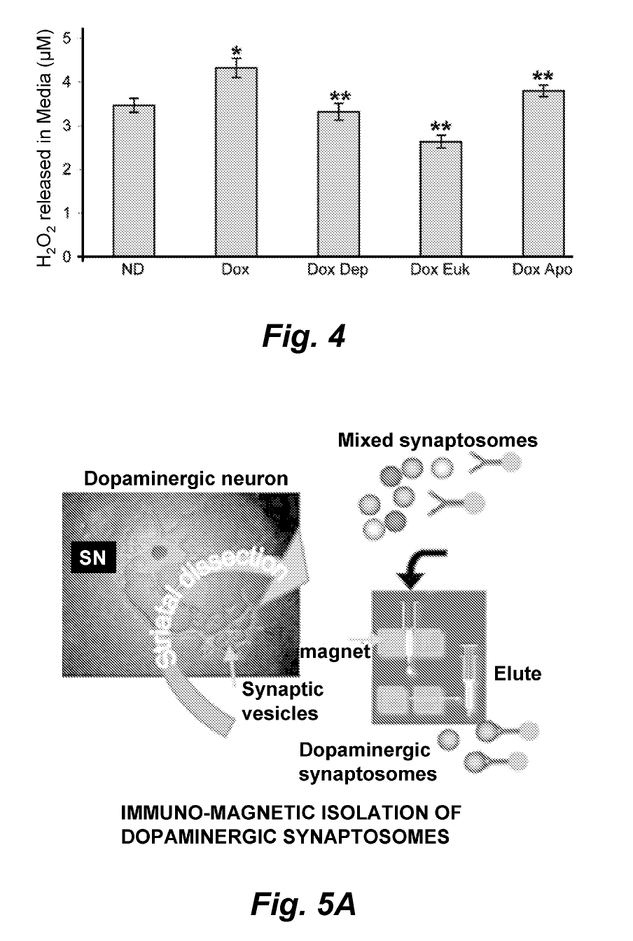

Based on our data, global increases in astrocytic MAO-B appeared to result in a preferential neurodegeneration of dopaminergic neurons suggesting that dopamine itself may be involved in the neurodegenerative process. Recent work in isolated brain mitochondria [34] demonstrated that dopamine oxidized to dopaminochrome (DACHR) can remove electrons from the auto-oxidizable site in mitochondrial complex I forming dopaminochrome radical. Dopaminochrome radical can in turn react with O₂ present in the mitochondria converting it to O₂⁻ in concert with re-reduction of the dopaminergic radical, setting up a redox cycling event. This would be manifest as an increase in mitochondrial O₂⁻ levels and ROS-induced complex I inhibition within the dopaminergic neurons which could result in their selective demise. We hypothesized that membrane-permeant H₂O₂ produced by increased MAO-B activity within astrocytes could interact with intercellular dopamine and produce selective degenerative effects on dopaminergic neurons via a similar mechanism. To first test for the ability of astrocytes with elevated MAO-B activity to produce increased levels of extracellular ROS, we measured H₂O₂ generated in the culture medium in mesencephalic cultures isolated from our inducible MAO-B transgenics following dox induction. H₂O₂ levels released into the media in dox-induced cultures were significantly higher than those produced from untreated cultures (FIG. 4). Co-treatment with deprenyl prevented the increase in extracellular H₂O₂ as did treatment with the antioxidant superoxide dismutase-catalase mimetic compound EUK-1 89. Co-treatment with apocynin, an NADP11 oxidase inhibitor, resulted in a partial reduction in the H₂O₂ levels indicating that microglial activation contributes to the ROS increase although it is not clear whether this is directly due to astroglial activation or a secondary effect of dopaminergic cell death in the cultures. Iba1⁻ cells (a 17 kDa calcium binding protein expressed in microglia) were found to constitute 3% of the total cell population and their numbers were increased by ˜17% following dox induction (data not shown).

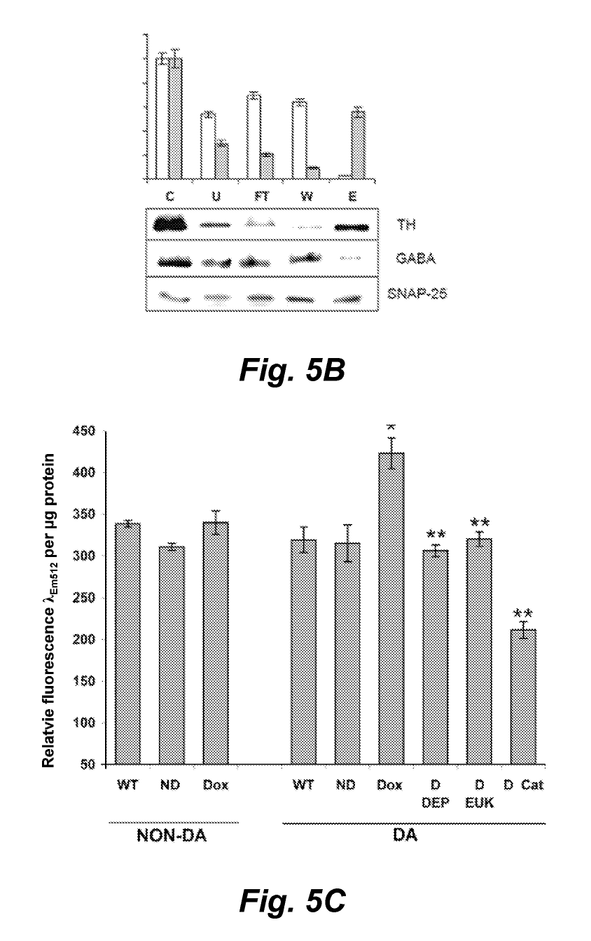

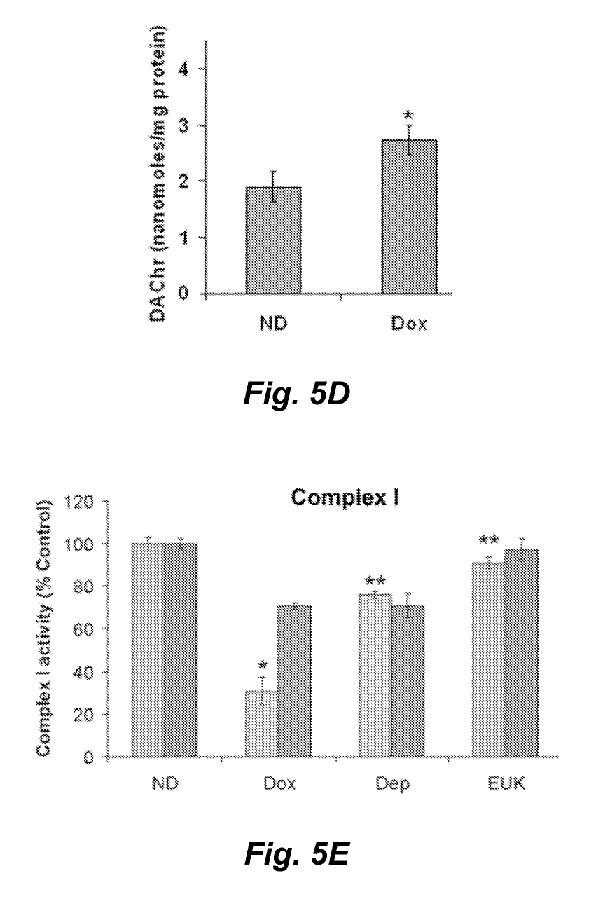

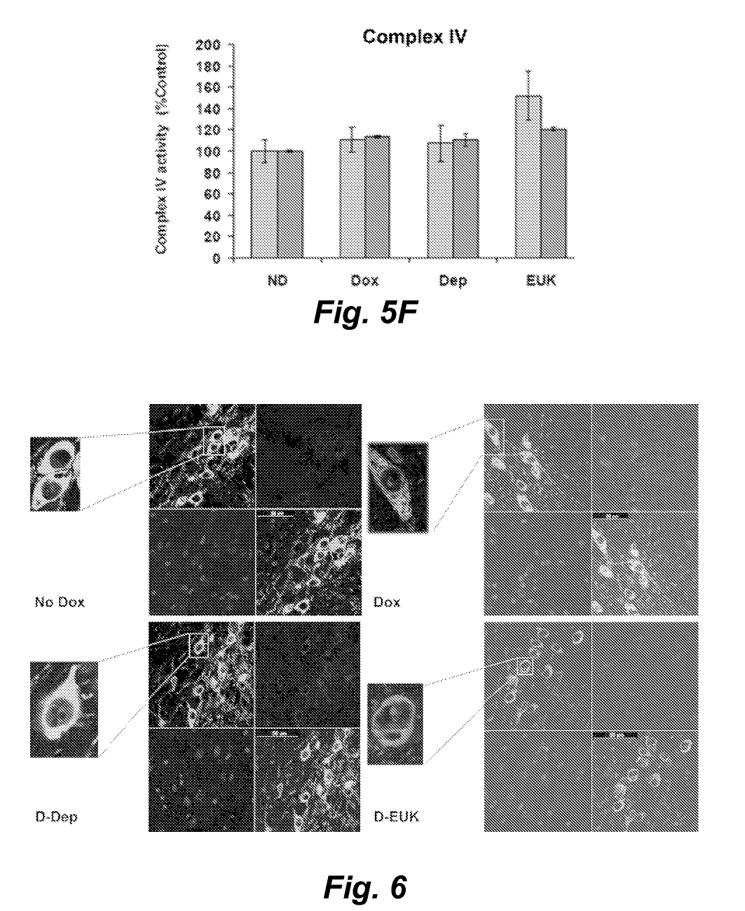

Dopaminergic neurons constitute only a minor portion (˜5%) of cells making up the SN (unpublished observations). In contrast to the SN, the striatum (ST) is larger, easier to dissect out, and at least 10-15% of the striatal nerve terminals originate from SN dopaminergic neurons [35-37]. In order to achieve meaningful data as to the impact of astrocytic MAO-B elevation on H₂O₂ levels and mitochondrial function selectively within dopaminergic nigrostriatal neurons, we chose to enrich for striatal dopaminergic nerve terminal (synaptosomal) populations using antibody against DAT via a modified immunomagnetic bead approach (FIGS. 5A, 5B). Synaptosomes in the flow-through fractions were non-dopaminergic (GABA⁺) and therefore used as negative controls in our subsequent biochemical analyses.

H₂O₂ levels were measured in isolated ST dopaminergic synaptosomes following injection of DCF into the tail vein of dox-induced versus uninduced and wildtype littermate controls as modified from Andrews et al. [38]. H₂O₂ levels was found to be elevated in isolated ST dopaminergic synaptosomes following increased astrocytic MAO-B elevation; this was found to be preventable by co-treatment of animals with either deprenyl or EUK-189 (FIG. 5C). Catalase treatment of the dopaminergic synaptosomes from the induced animals reduced the fluorescence to below uninduced levels indicating that the DCF fluorescence is due to H₂O₂. In conjunction with elevations in H₂O₂ within nerve terminals originating from dopaminergic SN neurons, we found an elevation in striatal DAC11R. DAC11R is a stable derivative of dopamine quinone, the major species produced by dopamine oxidation [39] (FIG. 5D). Along with increases in DAC11R levels, we found a preferential reduction in rotenone-inhibitable complex I (CI) activity in the ST dopaminergic versus non-dopaminergic synaptosomes (FIG. 5). In contrast, no change in complex IV activity was observed in either population following doxinduction (FIG. 5E). Loss of CI activity was attenuated in dopaminergic synaptosomes isolated from dox-induced animals that had been co-treated with deprenyl or EUK-189.

To assess whether increased dopaminergic H₂O₂ and DAC11R levels following astrocytic MAO-B elevation in vivo resulted in corresponding increases in mitochondrial O₂ levels within dopaminergic cells as previously demonstrated in vitro by Zoccarato and colleagues [34], animals were administered the superoxide indicator mitosox red via tail vein injection[38]. Dox-induced transgenics displayed elevated presence of mitosox red staining within mitochondria in both TH⁺ fibers in the striatum and in TH₊ neurons within the SN versus uninduced controls (FIG. 6, Table 2). Increases in mitosox red fluorescence within the dopaminergic SN neurons were prevented by co-treatment with either deprenyl or EUK1 89 (FIG. 6, Table 2). Significantly, dopaminergic SN cell loss was found to be attenuated in the presence of co-treatment with not only deprenyl, but also EUK1 89 suggesting that oxidative stress is an important component of dopaminergic demise (FIG. 3A).

TABLE 2
Superoxide in the SN dopaminergic neurons.

| | Mean pixel intensity | Total pixel fluorescence |
|---|---|---|
| | per cell | per cell |
| ND | 10.8 ± 2.0 | 101,983 ± 17,686 |
| D | 24.2 ± 3.6 | 223,526 ± 28,225 |
| DD | 10.2 ± 2.1 | 91,937 ± 19,107 |
| DEUK | 15.4 ± 4.0 | 126,757 ± 23,798 |

Increases in Astrocytic MAO-B Activity Results in Increased Local Microglial Activation in the SN.

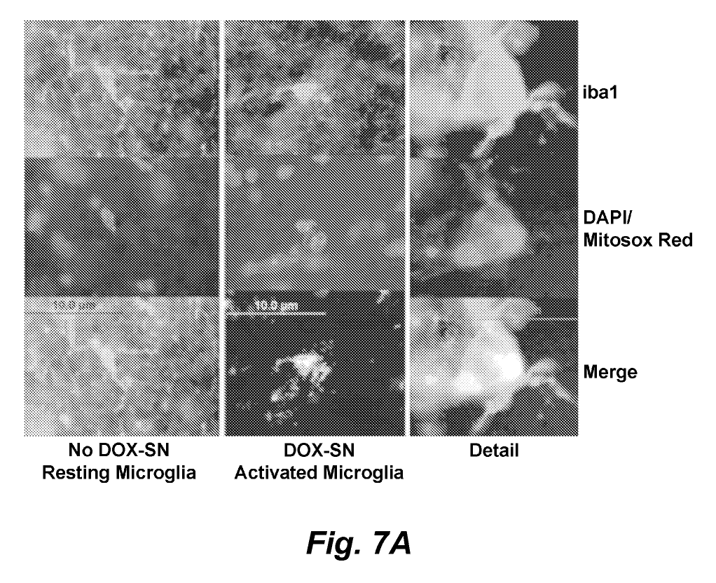

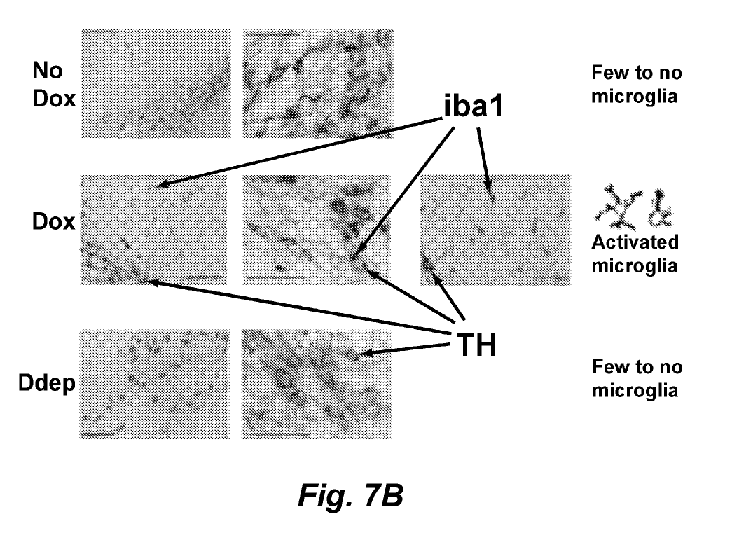

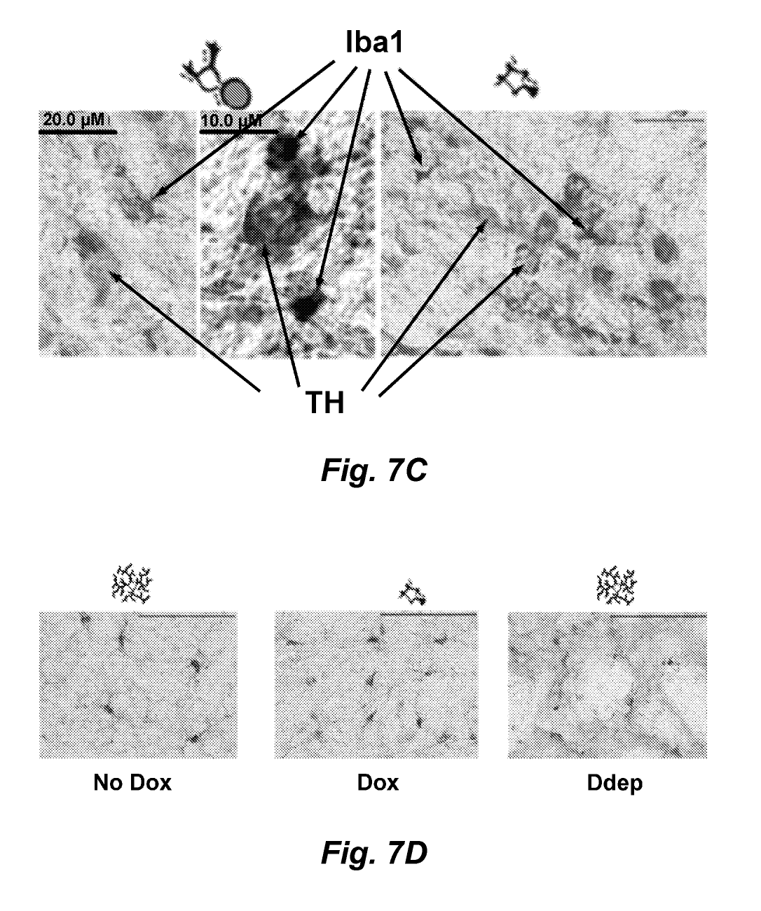

Data from our mitosox red studies not only suggested that SN dopaminergic neurons display an increase in oxidative stress following astrocytic MAO-B elevation, but also in the activated local SN microglial cells (FIG. 7A). This is likely a secondary effect subsequent to dopaminergic SN neurodegeneration triggering local microgliosis. Degeneration of SN dopaminergic neurons in PD has been observed to be accompanied by local microglial activation [40]. It has also been noted post-mortem in both humans and primates exposed to MPTP [41,42]. It is detected in the chronic mouse rotenone model prior to appearance of the dopaminergic lesion [43] and during selective SN dopaminergic neurodegeneration in the spontaneous weaver mouse mutant [44]. We performed immunochemistry to evaluate microglial activation in our induced MAO-B transgenics via That immunostaining [45]. Prior to activation, microglia normally exhibit a highly ramified morphology. In response to an activating signal, microglia begin to withdraw their ramified branches and to extend new protrusions. Next they begin migration through the tissue, engulfing cells [46]. Astrocytic MAO-B expression was found to lead to local microglial activation in both the SN and the striatum (FIG. 7 BD) but not the cortex (supplement FIG. S1). Unactivated ramified microglia were found in the uninduced nigrostriata and cortex; activated microglia with reduced branching in locomotory stages were only present within the SN and striatum of induced MAOB transgenics. In some cases, the microglia were in a high motile state with minimal processes or were in close contact with TH⁺ neurons, possibly in the phagocytic stage (FIG. 7C). Microglial activation was prevented by treatment with deprenyl (FIG. 7B). Co-treatment of animals with an inhibitor of microglial activation, minocycline, prevented dopaminergic SN cell death suggesting that microglial activation plays a major role in dopaminergic neurodegeneration in this model (FIG. 3A).

Increases in Astrocytic MAO-B Activity Results in Decreased Locomotor Movement.

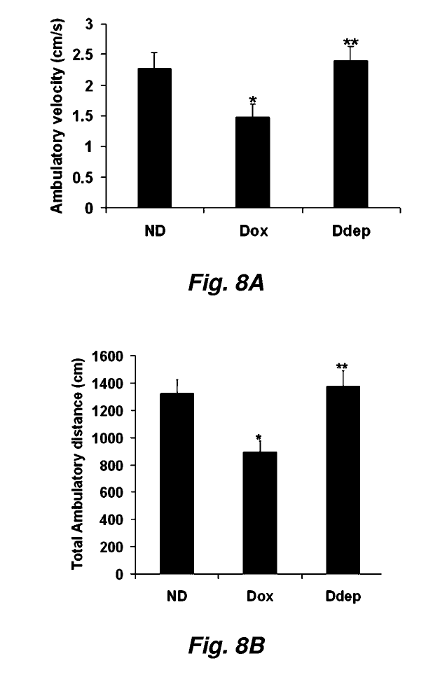

Finally, astrocytic increases in MAO-B in our model were found to correlate with a significant inhibition of locomotor function. Open field analysis of dox-treated mice revealed a significant difference in locomotor behavior in induced versus uninduced transgenics (FIG. 8A, B). After treatment with dox alone for two weeks, induced mice displayed a ˜32-35% decrease in locomotor activity in comparison to uninduced littermates. In contrast, deprenyl-treated induced mice were completely unaffected.

### Discussion

Increased brain MAO-B levels have been hypothesized to play a role in neuropathies associated with PD [47-5 1] however direct proof of a causative role has been thus far lacking. In this study, we demonstrate that elevations in astrocytic MAO-B levels results in a relatively selective loss of dopaminergic SN neurons and the severity of this loss appears to be age-dependent. It is not clear why there was not a more dramatic increase in SN DA cell loss with age in the MAO-B transgenics. Normally, cell loss coincides with an age-related increase in MAO-B enzyme activity. Our results suggest that the elevation of MAO-B to aging levels in the young animals was sufficient on its own to produce significant cell loss and that the additional stress of an aging brain contributed marginally (yet significantly) to this effect. Observed cell loss was accompanied by increased mitochondrial oxidative stress and selective decreases in mitochondrial complex I activity in these cells along with local microglial activation all of which we assume contribute to subsequent cell death. These pathological alterations were found to correlate with a significant decrease in locomotory behavior. Our data, taken in total, demonstrates that elevations in levels of astrocytic MAO-B activity results in several of key pathological hallmarks of Parkinson's disease.

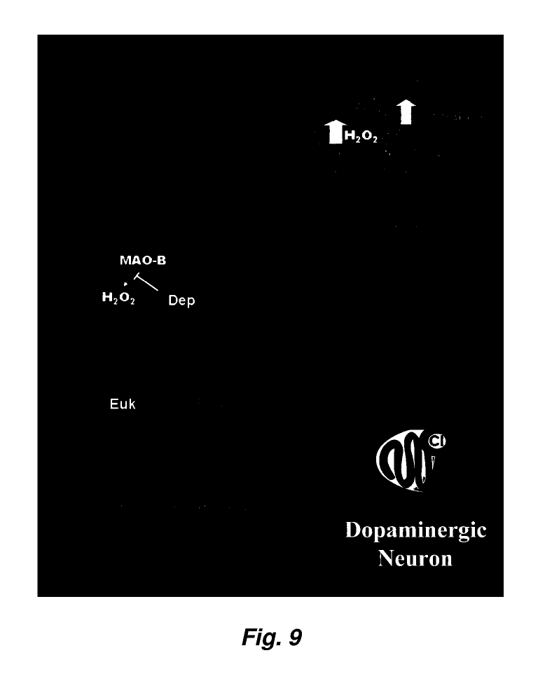

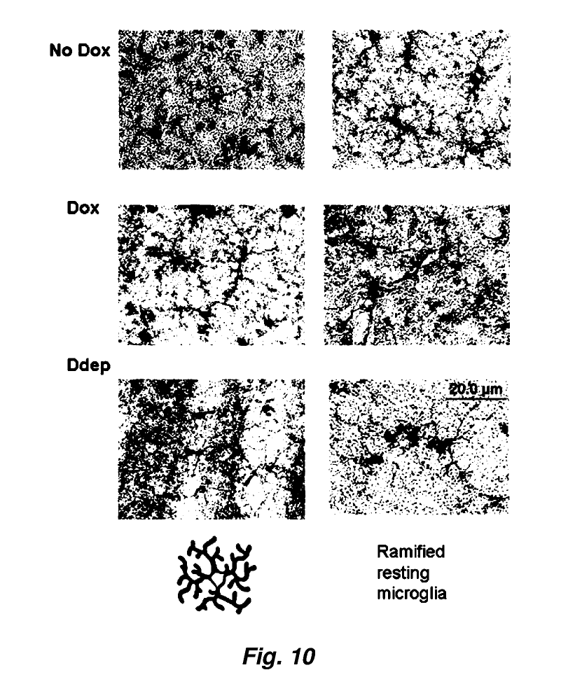

ROS produced by MAO-B-expressing astrocytes can be released into the extracellular environment and, due to its high membrane permeability, diffuse into neighboring cells. Within dopaminergic neurons, it can oxidize dopamine to DACHR which can interact with electrons at the auto-oxidizable site of mitochondrial complex I at a higher affinity than oxygen itself producing DACR radical. Electrons can then be transferred from DACR radical to oxygen resulting in re-reduction of DACR to DACHR and production of superoxide as part of an ongoing redox cycling event. These events are shown schematically in FIG. 9. We have demonstrated in this current study that increased astrocytic MAO-B results in elevations in extracellular ROS levels and increased intracellular H₂O₂ within the terminals of SN dopaminergic neurons accompanied by increased dopamine oxidation to DAC11R, selective mitochondrial complex I inhibition and elevations in mitochondrial superoxide levels. These events are prevented by either co-treatment with the MAO-B inhibitor deprenyl or the antioxidant compound EUK189 which also prevented subsequent dopaminergic SN cell loss.

Another prominent feature noted in our model was local microglial activation within the nigrostriatum. Dramatic local proliferation of microglial cells has been reported in the post-mortem PD brain as well as in various animal models of the disease [40, 43, 44, 52-54]. Local microglial activation was found to be associated in our model with degenerating T11⁺ cells in both the SN and the striatum. It may be a secondary reaction to initiation of dopaminergic neurodegeneration. Significantly, dopaminergic SN cell loss is substantially attenuated by co-treatment of animals with the microglial activation inhibitor minocycline suggesting that microglial activation plays a significant role in dopaminergic demise associated with this model.

Taking in total, our data demonstrates a linkage between several separate pathological events associated with Parkinson's disease that have not necessarily been considered to be mechanistically connected including mitochondrial dysfunction, and microglial activation. This indicates that MAO-B is a common initiator for these events and provides a novel model for exploring the mechanisms by which these events can occur in the context of the human condition.

### REFERENCES

1. Westlund et al. (1988) Neuroscience 25: 439-456.
2. Westlund et al. (1985) Science 230: 181-183.
3. Levitt et al. (1982) Proc. Natl. Acad. Sci., USA, 79: 6385-6389.
4. Saura et al. (1994) J Neural Transm Suppl 41: 89-94.
5. Fowler et al. (1980) J Neural Transm 49: 1-20.
6. Riederer et al. (1987) Adv Neurol 45: 111-118.
7. Gerlach et al. (1996) Neurology 47: S137-145.
8. Vindis et al. (2001) Kidney Int 59: 76-86.
9. Cohen et al. (1997) Proc. Natl. Acad. Sci., USA, 94: 4890-4894.
10. Adams and Odunze (1991) Free Radic Biol Med 10: 161-169.
11. Halliwell (1992) J Neurochem 59: 1609-1623.
12. Raps et al. (1989) Brain Res 493: 398-401.
13. Sagara et al. (1993) J Neurochem 61: 1672-1676.
14. Makar et al. (1994) J Neurochem 62: 45-53.
15. Kang et al. (1997) Neuroreport 8: 2053-2060.
16. Buckman et al. (1993) J Neurochem 60: 2046-2058.
17. Behl et al. (1994) Cell 77: 8 17-827.
18. Whittemore et al. (1994) Neuroreport 5: 1485-1488.
19. Wei et al. (1996) J Neurosci Res 46: 666-673.
20. Damier et al. (1996) Neurology 46: 1262-1269.
21. Soong et al. (1992) Nat Genet. 2: 318-323.
22. Kumar et al. (2003) J Biol Chem 278: 46432-46439.
23. Betarbet et al. (2000) Nat Neurosci 3: 1301-1306.
24. Przedborski and Vila (2003) Ann N Y Acad Sci 991: 189-198.
25. Gainetdinov et al. (1997) J Neurochem 69: 1322-1325.
26. Wu et al. (1996) Ann N Y Acad Sci 786: 379-390.
27. Magyar and Szende (2004) Neurotoxicology 25: 23 3-242.
28. Szende et al. (2001) J Neural Transm 108: 25-33.
29. Shoulson (1992) The Parkinson Study Group. Eur Neurol 32 Suppl 1: 46-53.
30. LeWitt (1994) J Neural Transm Suppl 43: 171-181.
31. Schneider (1995) J Neural Transm Suppl 46: 39 1-397.
32. Parkinson Study Group (1998) Ann Neurol 43: 3 18-325.
33. Fearnley and Lees (1991) Brain 114 (Pt 5): 2283-2301.
34. Zoccarato (2005) J Biol Chem 280: 15587-15594.
35. Pickel et al. (1992) Brain Res Dev Brain Res 70: 75-86.
36. Nirenberg et al. (1996) J Neurosci 16: 4135-4145.
37. Nirenberg et al. (1996) J Neurosci 16: 436-447.
38. Andrews et al. (2005) J Neurosci 25: 184-191.
39. Ochs et al. (2005) J Neurosci Methods 142: 201-208.
40. McGeer et al. (1988) Neurology 38: 1285-1291.
41. Langston et al. (1999) Ann Neurol 46: 598-605.
42. McGeer et al. (2003) Ann Neurol 54: 599-604.
43. Sherer et al. (2003) J Neurosci 23: 10756-10764.
44. Peng et al. (2006) J Neurosci 26: 11644-1165 1.
45. Imai and Kohsaka (2002) Glia 40: 164-174.
46. Stence et al. (2001) Glia 33: 256-266.
47. Youdim and Bakhle (2006) Br J Pharmacol 147 Suppl 1: S287-296.
48. Mandel et al. (2003) CNS Drugs 17: 729-762.
49. Irwin et al. (1992) Brain Res 572: 224-231.
50. Cohen (1983) J Neural Transm Suppl 19: 89-103.
51. Olanow (1993) Mov Disord 8 Suppl 1: S1-7.
52. Cicchetti et al. (2002) Eur J Neurosci 15: 99 1-998.
53. Schneider and Denaro (1988) J Neuropathol Exp Neurol 47: 452-458.
54. Sugama et al. (2003) Brain Res 964: 288-294.
55. Miller (1972) Experiments in Molecular Genetics. Cold Spring Harbor, N.Y.: Cold Spring Harbor Laboratory Press.
56. Kaur et al. (2003) Neuron 37: 899-909.
57. Smith and Friedmann (2004) Neuroreport 15: 1025-1028.
58. Docherty et A (1991) J Neurochem 56: 1569-1580.
59. Budd and Nicholls (1995) J Neurochem 65: 615-621.
60. Trounce et al. (1996) Methods Enzymol 264: 484-509.
61. Peng et al. (2002) J Biol Chem 277: 44285-44291. J Biol Chem 279: 12406-12413.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

SEQUENCE LISTING

SEQ ID NO: 1 GenBank NM_000898 2611 bp mRNA linear PRI 11 Feb. 2008
DEFINITION Homo sapiens monoamine oxidase B (MAOB), nuclear gene
encoding mitochondrial protein, mRNA.
ORIGIN

| | |
|---|---|
| 1 | gcgcgtccgg gctcccgggg ctggtaatat agcggctcgc cgaggcgctg gtgcacgggg |
| 61 | gcagcgcgca gcaggccggc gggcaggcgg gcgggctggc tggcaggcag gactgggatc |
| 121 | gaggcccaga aaacggagca gcgggcacca gggaggcctg gaacggggcg agcgccatga |
| 181 | gcaacaaatg cgacgtggtc gtggtggggg gcggcatctc aggtatggca gcagccaaac |
| 241 | ttctgcatga ctctggactg aatgtggttg ttctggaagc ccgggaccgt gtgggaggca |
| 301 | ggacttacac tcttaggaac caaaaggtta aatatgtgga ccttggagga tcctatgttg |
| 361 | gaccaaccca gaatcgtatc ttgagattag ccaaggagct aggattggag acctacaaag |
| 421 | tgaatgaggt tgagcgtctg atccaccatg taaagggcaa atcatacccc ttcagggggc |
| 481 | cattcccacc tgtatggaat ccaattacct acttagatca taacaacttt tggaggacaa |
| 541 | tggatgacat ggggcgagag attccgagtg atgccccatg gaaggctccc cttgcagaag |
| 601 | agtgggacaa catgacaatg aaggagctac tggacaagct ctgctggact gaatctgcaa |
| 661 | agcagcttgc cactctcttt gtgaacctgt gtgtcactgc agagacccat gaggtctctg |
| 721 | ctctctggtt cctgtggtat gtgaagcagt gtggaggcac aacaagaatc atctcgacaa |
| 781 | caaatggagg acaggagagg aaatttgtgg gcggatctgg tcaagtgagt gagcggataa |
| 841 | tggacctcct tggagaccga gtgaagctgg agaggcctgt gatctacatt gaccagacaa |
| 901 | gagaaaatgt ccttgtggag accctaaacc atgagatgta tgaggctaaa tatgtgatta |
| 961 | gtgctattcc tcctactctg ggcatgaaga ttcacttcaa tccccctctg ccaatgatga |
| 1021 | gaaaccagat gatcactcgt gtgcctttgg gttcagtcat caagtgtata gtttattata |
| 1081 | aagagccttt ctggaggaaa aaggattact gtggaaccat gattattgat ggagaagaag |
| 1141 | ctccagttgc ctacacgttg gatgatacca aacctgaagg caactatgct gccataatgg |
| 1201 | gatttatcct ggcccacaaa gccagaaaac tggcacgtct taccaaagag gaaaggttga |
| 1261 | agaaactttg tgaactctat gccaaggttc tgggttccct agaagctctg gagccagtgc |
| 1321 | attatgaaga aaagaactgg tgtgaggagc agtactctgg gggctgctac acaacttatt |
| 1381 | tcccccctgg gatcctgact caatatggaa gggttctacg ccagccagtg gacaggattt |
| 1441 | actttgcagg caccgagact gccacacact ggagcggcta catggagggg gctgtagagg |
| 1501 | ccggggagag agcagcccga gagatcctgc atgccatggg gaagattcca gaggatgaaa |
| 1561 | tctggcagtc agaaccagag tctgtggatg tccctgcaca gcccatcacc accacctttt |
| 1621 | tggagagaca tttgccctcc gtgccaggcc tgctcaggct gattggattg accaccatct |
| 1681 | tttcagcaac ggctcttggc ttcctggccc acaaaagggg gctacttgtg agagtctaaa |
| 1741 | gagagagggt gtctgtaatc acactctctt cttactgtat ttgggatatg agtttgggga |
| 1801 | aagagttgca gtaaagttcc atgaagacaa atagtgtgga gtgaggcggg gagcatgaag |
| 1861 | ataaatccaa ctctgactgt aaaatacatg gtatctcttt ctccgttgtg gcccctgctt |
| 1921 | agtgtccctt acctggctta gcgttctgtt tcaccagttt ccaagtttat tgccctcaaa |
| 1981 | atctttagaa tagttaaatt ggcttgttta aggttcttgc tgccccacaa cacaccttgc |
| 2041 | ccatgcacaa ggaatgaatt ttttcctacc attatggctt tgtgcttgtt cttcctctta |
| 2101 | cctgtaatag cctcaccttc cctagttctt tgcattcgtc cttagaatac tgtattgtta |
| 2161 | cagctgaaag acagtaaaga ccatttagtc ctcaccttct gttttagagt tgagcaaact |
| 2221 | gaagcccaca gaggtggaac ttaattacct aagagccaca ataagccact ggtatctggg |
| 2281 | ggactagaac acaaatccaa cgcttttccc acctctttgg atgttttccc caattatcct |
| 2341 | ccttcactcc ctgtcatagt taccgatggt gtcccgttgt gtgggtttac tctgtgctaa |
| 2401 | gttgtcttac acttctcaaa tgctactcag tatatagcct taagtcttac tgttttgtgc |
| 2461 | ggtgtgtctc cagctgattt taactttttt gatggtagaa attttatctc ttcttccttt |
| 2521 | tgtatcctcc attgtatctt catacaaagg acagtacaca cttgggtaat taaaaataaa |
| 2581 | agttgattga ccataaaaaa aaaaaaaaaa a |

SEQ ID NO: 2
/translation = “MSNKCDVVVVGGGISGMAAAKLLHDSGLNVVVLEARDRVGGRTY
TLRNQKVKYVDLGGSYVGPTQNRILRLAKELGLETYKVNEVERLIHHVKGKSYPFRGP
FPPVWNPITYLDHNNFWRTMDDMGREIPSDAPWKAPLAEEWDNMTMKELLDKLCWTES
AKQLATLFVNLCVTAETHEVSALWFLWYVKQCGGTTRIISTTNGGQERKFVGGSGQVS
ERIMDLLGDRVKLERPVIYIDQTRENVLVETLNHEMYEAKYVISAIPPTLGMKIHFNP
PLPMMRNQMITRVPLGSVIKCIVYYKEPFWRKKDYCGTMIIDGEEAPVAYTLDDTKPE
GNYAAIMGFILAHKARKLARLTKEERLKKLCELYAKVLGSLEALEPVHYEEKNWCEEQ
YSGGCYTTYFPPGILTQYGRVLRQPVDRIYFAGTETATHWSGYMEGAVEAGERAAREI
LHAMGKIPEDEIWQSEPESVDVPAQPITTTFLERHLPSVPGLLRLIGLTTIFSATALG
FLAHKRGLLVRV”

1. A method of identifying a mammal at risk for Parkinson's disease, said method comprising:
determining level of expression or activity of monoamine oxidase B (MAO-B) in a sample from said mammal wherein an elevated level of MAO-B expression and/or activity as compared to a control is an indicator that said mammal has an increased likelihood of developing Parkinson's disease.

2. The method of claim 1, wherein said mammal is asymptomatic for Parkinson's disease.

3. The method of claim 1, wherein said mammal presents with no significant neuronal cell loss.

4. The method of claim 1, wherein said mammal is a human.

5. The method of claim 1, wherein said mammal is a human that presents without symptoms of Parkinson's disease, but is believed to be at risk for the disease.

6. (canceled)

7. The method of claim 1, wherein said mammal is a human.

8. The method of claim 1, wherein said sample comprises one or more biological materials selected from the group consisting of blood or a blood fraction, platelets, saliva, cerebrospinal fluid, and a tissue sample.

9. The method of claim 1, wherein said control comprises an expression and/or activity level determined for a population of said mammal that does not develop Parkinson's disease.

10. The method of claim 9, wherein said population consists of members of the same species having the same age, and/or sex, and/or ethnicity.

11. The method of claim 1, wherein said control comprises a threshold value designated as indicative of increased risk for Parkinson's disease.

12. The method of claim 1, wherein said control is the level of MAO-B expression and/or activity in same subject at a different time in their life.

13. The method of claim 1, wherein said control, is the threshold of top 10% percentile of the full range of MAO-B activity within a random population.

14. The method of claim 1, wherein said method is a component of a differential diagnosis for Parkinson's disease.

15. The method of claim 1, wherein said method further comprises recording the determined level of MAO-B expression or activity, and/or a diagnosis based at least in part on the determined level of MAO-B expression or activity, in a patient medical record.

16. The method of claim 15, wherein said medical record is a medical record selected from the group consisting of a hospital medical record, a doctor's office medical record, an insurance company medical record, a health maintenance organization (hmo) medical record, a personal medical record, a laboratory medical record, a personal medical record website, a computer readable medium storing a medical record, and a radio frequency tag (RF tag).

17. The method of claim 15, wherein a diagnosis, based at least in part on the level of expression or activity of MAO-B is recorded on or in a medic alert article selected from a card, worn article, or radio frequency tag.

18. (canceled)

19. The method of claim 1, wherein said method further comprises one or more actions selected from the group consisting of prescribing, or initiating and/or altering prophylaxis and/or therapy for Parkinson's disease in said subject when said determining provides a positive result, scheduling the same test or a different test in a differential diagnostic protocol for Parkinson's disease, and scheduling follow-up diagnostics to monitor said subject for the onset of Parkinson's disease.

20-21. (canceled)

22. The method of claim 1, wherein
MAO-B nucleic acid and/or an MAO-B protein or fragment thereof is detected in an assay wherein the MAO-B protein or nucleic acid becomes labeled with a detectable label; and/or
MAO-B nucleic acid and/or a MAO-B protein or fragment thereof is detected in an assay wherein the MAO-B protein or nucleic acid is transformed from a free state to a bound state by forming a complex with another assay component; and/or
MAO-B nucleic acid and/or an MAO-B protein or fragment thereof is detected in an assay wherein a MAO-B nucleic acid and/or MAO-B protein or fragment thereof initially present in a soluble phase becomes immobilized on a solid phase; and/or
MAO-B nucleic acid and/or an MAO-B protein or fragment thereof is detected in an assay wherein the sample is fractionated to separate MAO-B protein or nucleic acid from at least one other sample component; and/or
MAO-B nucleic acid and/or a MAO-B protein or fragment thereof is detected in an assay wherein MAO-B nucleic acid or protein becomes embedded in a separation medium; and/or
MAO-B nucleic acid and/or a MAO-B protein or fragment thereof is detected in an assay wherein a MAO-B nucleic acid or protein is volatilized.

23-27. (canceled)

28. The method of claim 1, wherein said determining comprises determining activity of MAO-B in said sample.

29. The method of claim 28, wherein said determining comprises reacting MAO-B in said sample with a monamine substrate, a benzylamine substrate, a phenylethylamine substrate, a fluorogenic substrate, or a PET scan.

30-32. (canceled)

33. The method of claim 1, wherein said determining comprises determining MAO-B expression level.

34. The method of claim 33, wherein said determining comprises determining the level of an MAO-B mRNA or a nucleic acid derived therefrom in said sample.

35. The method of claim 34, wherein said determining comprises transforming the sample or fraction derived therefrom by amplifying a reverse transcript of MAO-B mRNA from said sample.

36. The method of claim 35, wherein said amplifying is by polymerase chain reaction (PCR).

37. (canceled)

38. The method of claim 33, wherein determining the expression level of an MAO-B nucleic acid comprises a method selected from the group consisting of a Northern blot, a Southern blot using DNA derived from a MAO-B RNA, an array hybridization, an affinity chromatography, and an in situ hybridization.

39-42. (canceled)

43. The method of claim 33, wherein said determining the expression level of MAO-B comprises determining the amount of a MAO-B protein in said sample.

44. The method of claim 43, wherein said determining is via a method selected from the group consisting of capillary electrophoresis, a Western blot, mass spectroscopy, ELISA, immunochromatography, and immunohistochemistry.

45-49. (canceled)

50. A method of prophylactically treating a subject for Parkinson's disease, said method comprising: identifying a subject whose level of expression or activity of monoamine oxidase B (MAO-B) is elevated as compared to a control indicating that said mammal has an increased likelihood of developing Parkinson's disease; and prescribing for said subject and/or administering to said subject one or more MOA-B inhibitors.

51. (canceled)

52. The method of claim 50, wherein said one more MAO-B inhibitors includes an inhibitor selected from the group consisting of selegiline, desmethylselegiline, pargyline, rasagiline, caroxyazone, AGN-1135, MDL 72195, J 508, lazabemide, milacemide, IFO, mofegiline, and 5-(4-(4,4,4-trifluorobut-oxy)phenyl)-3-(2-methoxyethyl)-1,3,4-oxadiazol-2(3H)-one.

53. The method of claim 50 where said MOA-B inhibitors exclude one or more agents selected from the group consisting of tumeric, cigarettes or component(s) thereof, tryptamines, and antibiotics

54. An animal model for Parkinson's disease, said model comprising a transgenic non-human mammal in which monoamine oxygenase B can be inducibly increased in astrocytes in the brain of said mammal.

55-58. (canceled)

59. A method of identifying an MAO-B inhibitor, said method comprising: administering to the animal model of claim 54, one or more test agents; and detecting the expression and/or activity of MAO-B in said test animal under conditions wherein the MAO-B transgene is expected to be expressed and/or active, where a reduction in expression and/or activity of MAO-B indicates that the test agent(s) are inhibitors of MAO-B.

60. The method of claim 59, wherein said detecting the expression and/or activity of MAO-B in said test animal under conditions wherein the MAO-B transgene is expected to be expressed and/or active comprises a method according to claim 1.

61. A kit for diagnosing a predisposition to Parkinson's disease, said kit comprising: a container containing a means for detecting MAO-B expression and/or activity; and instructional materials teaching the use of MAO-B expression and/or activity level(s) as a measure of risk for Parkinson's disease.
